(19) United States
(12) Patent Application Publication
(10) Pub. No.: US 2026/0207654 A1
(43) Pub. Date: Jul. 23, 2026

(54) HEPARAN SULFATE AND ITS MIMETICS AS CHEMOKINE INHIBITORS

(71) Applicant: INDIAN INSTITUTE OF SCIENCE EDUCATION AND RESEARCH, Pune (IN)

(72) Inventors: Raghavendra KIKKERI, Pune (IN); N. Vijendra KUMAR, Pune (IN); Chethan D SHANTHAMURTHY, Pune (IN); Prashant JAIN, Pune (IN); Rakesh RAIGAWALI, Pune (IN); Vered PADLER-KARAVANI, Aviv (IL); Shani LEVIATAN BEN-ARYE, Aviv (IL)

(21) Appl. No.: 18/262,463

(22) PCT Filed: Jan. 22, 2022
(86) PCT No.: PCT/IB2022/050566

§ 371 Date: Jul. 21, 2023

(30) Foreign Application Priority Data
Jan. 22, 2021 (IN) 202121003052

(51) Int. Cl. A61K 31/727; A61P 35/00; C08B 37/00

(52) U.S. Cl. CPC A61K 31/727; A61P 35/00; C08B 37/0075

(57) ABSTRACT

The present invention relates to a heparan sulfate compound of formula (I) & (II) or a pharmaceutically acceptable salt thereof and pharmaceutical composition containing them. The present invention also relates to a process for preparing the compound of formula (I) & (II) or a pharmaceutically acceptable salt thereof and use of the compound of formula (I) & (II) or a pharmaceutically acceptable salt thereof as chemokine inhibitors

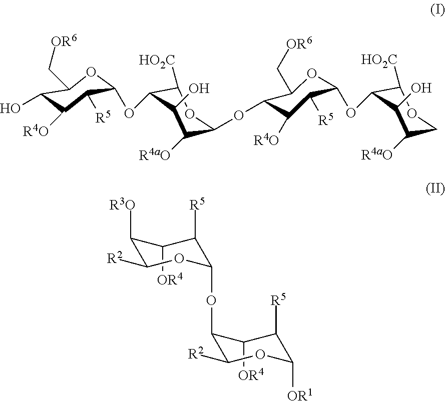

### FIELD OF THE INVENTION

The present invention generally relates to organic compounds. Specifically, the present invention relates to a heparan sulfate and its L-Iduronic acid based mimics compound of formula (I) & (II) or a pharmaceutically acceptable salt thereof and pharmaceutical composition containing them. The present invention also relates to a process for preparing the compound of formula (I) & (II) or a pharmaceutically acceptable salt thereof and use of the compound of formula (I) & (II) or a pharmaceutically acceptable salt thereof as chemokine inhibitors.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

Chemokines are a family of small proteins that have become a focus of extensive research due to their diverse roles in numerous physiological and pathological processes, including cell trafficking, angiogenesis, embryonic development, neurodegenerative diseases and cancer (Brylka, L. J.; Schinke, T. Chemokines in physiological and pathological bone remodeling, Front. Immunol., 2019, 10, 2182). Thus, selective inhibition of chemokines can be beneficial in controlling indications such as inflammation, viral entry, cancer progression etc., (Koenen, R. R.; Weber, C. Therapeutic targeting of chemokine interactions in atherosclerosis, Nat. Rev. Drug Discovery, 2010, 9, 141-153). As of now, only very few chemokine inhibitors have been developed and are under clinical evaluation (Andrews, S. P.; Cox, R. J. Small molecule CXCR3 antagonists. J. Med. Chem. 2016, 59, 2894-2917). Hence, there is a large unmet need for developing new and more potent chemokine inhibitors.

It has been shown that chemokines utilize the highly sulfated glycosaminoglycan (GAG) heparan sulfate (HS), chondroitin sulfate (CS), dermatan sulfate (DS), as a co-receptor to oligomerize and activate their cell surface receptors (Johnson, Z.; Proudfoot, A. E.; Handel, T. M. Interaction of chemokines and glycosaminoglycans: a new twist in the regulation of chemokine function with opportunities for therapeutic intervention. Cytokines Growth Factor Rev. 2005, 16, 625-636).

Gallagher et al. reported that the CXCL4 chemokine requires HS 2-O-sulfated iduronic acid (IdoA) for tetramerization and binding to its cell surface receptors (Stringer, S. E.; Gallagher, J. T., Specific binding of the chemokine platelet factor 4 to heparan sulfate. J. Biol. Chem. 1997, 272 (33), 20508-20514). Elsewhere, Lindahl et al. had shown that interleukin-8 (CXCL8 or IL-8) prefers the IdoA (2-OSO₃⁻)-GlcNSO₃(6-OSO₃⁻) repeating unit to activate neutrophil trafficking (Spillmann, D.; Witt, D.; Lindahl, U., Defining the interleukin-8-binding domain of heparan sulfate. J. Biol. Chem. 1998, 273 (25), 15487-15493), while Gardiner et al. reported the elegant role of 6-O-sulfation in switching the binding between CXCL12 and IL-8 (Jayson, G. C.; Hansen, S. U.; Miller, G. J.; Cole, C. L.; Rushton, G.; Avizienyte, E.; Gardiner, J. M., Synthetic heparan sulfate dodecasaccharides reveal single sulfation site interconverts CXCL8 and CXCL12 chemokine biology. Chem. Comm. 2015, 51 (72), 13846-13849). Hesieh-Wilson et al. demonstrated that the trisulfated IdoA (2-OSO₃⁻)-GlcNSO₃(6-OSO₃⁻)-conjugated polymer strongly inhibited RANTES (CCL5)-CCR3-receptor-mediated cell migration (Sheng, G. J.; Oh, Y. I.; Chang, S.-K.; Hsieh-Wilson, L. C., Tunable heparan sulfate mimetics for modulating chemokine activity. J. Am. Chem. Soc. 2013, 135 (30), 10898-10901). In addition, Seeberger et al., showed that CCL21 strongly binds to a hexasaccharide containing GlcNSO₃(6-OSO₃⁻)-IdoA(2-OSO₃⁻) repeating unit as compared to CXCL12, while CCL19 does not bind to it at all (de Paz, J. L.; Moseman, E. A.; Noti, C.; Polito, L.; von Andrian, U. H.; Seeberger, P. H., Profiling heparin-chemokine interactions using synthetic tools. ACS Chem. Biol. 2007, 2 (11), 735-744). Boons et al. discovered that CCL2 binds to highly sulfated HS compounds and exhibits no preference for the uronic acid component, while both CCL2 and CCL13 displayed promiscuous binding with most of the HS glycans (Zong, C.; Venot, A.; Li, X.; Lu, W.; Xiao, W.; Wilkes, J.-S. L.; Salanga, C. L.; Handel, T. M.; Wang, L.; Wolfert, M. A., Boons, G. J., Heparan sulfate and its mimics microarray reveals that heparan sulfate-protein binding exhibits different ligand requirements. J. Am. Chem. Soc. 2017, 139 (28), 9534-9543).

Heparan sulfate (HS) compounds available in the prior arts are highly heterogeneous in its structure thereby rendering the HS compounds less specific for chemokine inhibition.

Therefore, there is an unmet need for new HS compounds that are more specific for chemokine inhibition.

### OBJECTS OF THE INVENTION

An object of the present invention is to provide novel compounds and a process for preparation thereof.

Another object of the present invention is to provide a compound of formula (I) & (II) that is more specific for chemokine inhibition.

Another object of the present invention is to provide a compound of formula (I) & (II) having anticancer activity.

### SUMMARY OF THE INVENTION

The present invention generally relates to organic compounds. Specifically, the present invention relates to heparan sulfate and its L-Iduronic acid based mimics compound of formula (I) & (II) or a pharmaceutically acceptable salt thereof and pharmaceutical composition containing them. The present invention also relates to a process for preparing the compound of formula (I) & (II) or a pharmaceutically acceptable salt thereof and use of the compound of formula (I) & (II) or a pharmaceutically acceptable salt thereof as chemokine inhibitors.

In a first aspect, the present invention relates to a heparan sulfate and its L-Iduronic acid based mimics compound of formula (I) or a stereoisomer, a tautomer, a conformer, a pharmaceutically acceptable salt or a pharmaceutically acceptable solvate thereof;

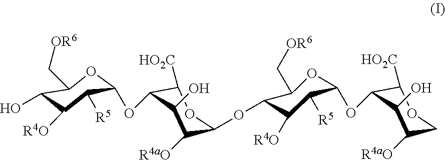

wherein:
R¹ is —(CH₂)₃NH₂;
R⁴ is H or SO₃⁻;
R^{4a} is H or SO₃⁻;
R⁵ is NH₂ or NHCOCH₃; and
R⁶ is H, SO₃⁻ and PO₄²⁻.

In another embodiment of the present invention, the compound of formula (I) encompasses a compound of formula (Ia) or a stereoisomer, a epimer, a conformer, a pharmaceutically acceptable salt or a pharmaceutically acceptable solvate thereof,

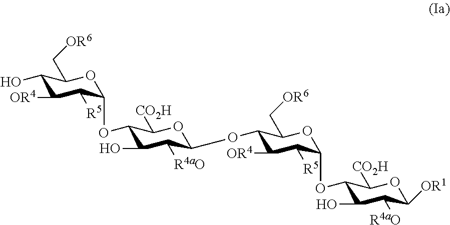

wherein:
R¹ is —(CH₂)₃NH₂;
R⁴ is H or SO₃⁻;
R^{4a} is H or SO₃⁻;
R⁵ is NH₂ or NHCOCH₃; and
R⁶ is H, or SO₃⁻.

In another aspect, the present invention relates to a heparan sulfate compound of formula (II) or a stereoisomer, a tautomer, a conformer, a pharmaceutically acceptable salt, a homooligosaccharides or a pharmaceutically acceptable solvate thereof,

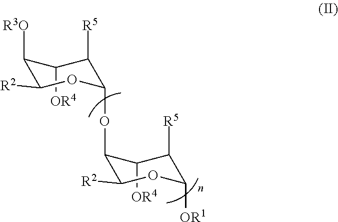

wherein:
R¹ is

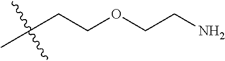 [SMILES: *CCOCCN]

or cholestenol;
R² independently represents COOH or CH₂OH;
R³ independently represents H or SO₃⁻;
R⁴ independently represents H, or SO₃⁻;
R⁵ independently represents OH, or OSO₃⁻,
n is 0, 1, 2 or 3.

In another aspect, the present invention relates to a heparan sulfate compound of formula (II) or a stereoisomer, a tautomer, a conformer, a pharmaceutically acceptable salt, a homooligosaccharides or a pharmaceutically acceptable solvate thereof,

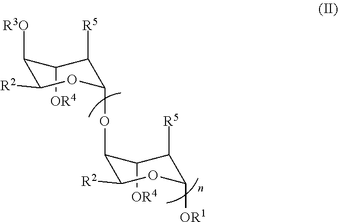

wherein:
R¹ is

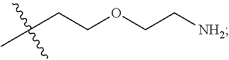 [SMILES: *CCOCCN]

R² independently represents COOH;
R³ independently represents H or SO₃⁻;
R⁴ independently represents H, or SO₃⁻;
R⁵ independently represents OH, or OSO₃⁻,
n is 0, 1, 2 or 3.

In another aspect, the present invention relates to a heparan sulfate mimics compound of formula (II) or a hydrophobic moieties, a conformer, a pharmaceutically acceptable salt, a homooligosaccharides or a pharmaceutically acceptable solvate thereof,

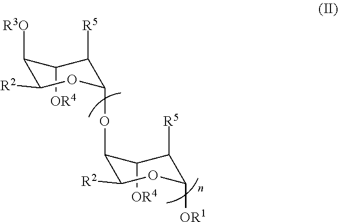

R¹ is cholestenol;
R² independently represents COOH;
R³ independently represents SO₃⁻;
R⁴ independently represents H;
R⁵ independently represents OSO₃⁻; and
n is 1, 2 or 3.

In one aspect, the present invention relates to a heparan sulfate mimics compound of formula (II) or a hydrophobic moieties, a idose, a pharmaceutically acceptable salt, a homooligosaccharides or a pharmaceutically acceptable solvate thereof,

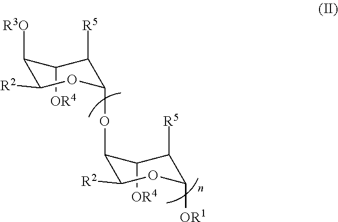

R¹ is cholestenol;
R² independently represents CH₂OH;
R³ independently represents SO₃⁻;
R⁴ independently represents H;
R⁵ independently represents OSO₃⁻; and
n is 1, 2, or 3.

In another aspect, the present invention relates to a pharmaceutical composition comprising a compound of formula (I) & (II) along with one or more pharmaceutically acceptable excipients.

In another aspect, the present invention relates to a heparan sulfate compound of formula (I) & (II) or a stereoisomer, a tautomer, a conformer, a pharmaceutically acceptable salt, or a pharmaceutically acceptable solvate thereof for use in the treatment of cancer.

In another aspect, the present invention relates to a method of treating a cancer comprising administering a therapeutically effective amount of heparan sulfate compound of formula (I) & (II) or a stereoisomer, a tautomer, a conformer, a pharmaceutically acceptable salt or a pharmaceutically acceptable solvate thereof to a subject in need thereof.

Various objects, features, aspects and advantages of the inventive subject matter will become more apparent from the following detailed description of preferred embodiments.

### BRIEF DESCRIPTION OF THE DRAWINGS

FIG. 1: SPR analysis of chemokines binding profile for the compounds of present invention: SPR binding analysis of the interaction between the examples 12, 17, and 19, chemokines were reported, Concentrations of chemokines were 0.05-2 μM. A global fit according to a 1:1 binding model was applied (black curves).

FIG. 2: MCF-7 and MDA-MB-231 cell proliferation was quantified by WST assay after 48 and 72 hrs treatment with the examples 11, 12 and 17 at different concentration with CCL2 chemokine.

FIG. 3 depicts MCF-7 cell cycle progress in the presence and absence of examples 11 and 12.

FIG. 4 shows cell migration assay: Area repopulated in the presence of example 11, 12, 17 with CCL2 chemokine is considered as 100% wound heal and data expressed as mean±SD (n=3; * P<0.05, *P<0.01).

FIG. 5: Boyden chamber assay was performed in presence of the examples 11, 12, 17 and Hep (50 μg ml⁻¹) with or without CCL2 (50 ng)

FIG. 6 relates to MAPK pathway analysis: MCF-7 cells were treated with CCL2 (50 ng) with or without the examples 11, 12, 17 ligands (50 μg/ml) and cell lysate was prepared at 30 min time points and P-p44/42 and total p44/42 was imaged.

### DETAILED DESCRIPTION OF THE INVENTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Reference throughout this specification to “one embodiment” or “an embodiment” or “another embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context requires otherwise, throughout the specification which follow, the word “comprise” and variations thereof, such as, “comprises” and “comprising” are to be construed in an open, inclusive sense that is as “including, but not limited to.”

Also, use of “(s)” as part of a term, includes reference to the term singly or in plurality, for example, the term pharmaceutically acceptable salt(s) indicates a single salt or more than one salt of the compound of formula (I) & (II).

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description that follows, and the embodiments described herein, is provided by way of illustration of an example, or examples, of particular embodiments of the principles and aspects of the present disclosure. These examples are provided for the purposes of explanation, and not of limitation, of those principles and of the disclosure.

It should also be appreciated that the present disclosure can be implemented in numerous ways, including as a system, a method or a device. In this specification, these implementations, or any other form that the invention may take, may be referred to as processes. In general, the order of the steps of the disclosed processes may be altered within the scope of the invention.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

Unless otherwise indicated, the following definitions are set forth to illustrate and define the meaning and scope of the various terms used to describe the invention herein and the appended claims. These definitions should not be interpreted in the literal sense as they are not intended to be general definitions and are relevant only for this application.

The term “or”, as used herein, is generally employed in its sense including “and/or” unless the content clearly dictates otherwise.

The term, “therapeutically effective amount” as used herein refers to an amount of a compound of formula (I) or a pharmaceutically acceptable salt thereof or a composition comprising a compound of formula (I) or a salt thereof, effective in producing the desired therapeutic response in a particular patient (subject) suffering from a disease or disorder.

The term “pharmaceutically acceptable excipient(s)” as used herein refers to a diluent, binder, disintegrant, glidant, lubricant, coating material or the like, which is non-toxic, and inert, which does not have undesirable effects on a subject to whom it is administered and is suitable for delivering a therapeutically active agent to the target site without affecting the therapeutic activity of the said agent.

The term, “subject” as used herein refers to an animal, preferably a mammal, and most preferably a human. The term “mammal” used herein refers to warm-blooded vertebrate animals of the class ‘mammalia’, including humans, characterized by a covering of hair on the skin and, in the female, milk-producing mammary glands for nourishing the young, the term mammal includes animals such as cat, dog, rabbit, bear, fox, wolf, monkey, deer, mouse, pig and human.

The terms, “treatment”, “treat” and “therapy” and the like as used herein refer to alleviate, slow the progression, attenuation, prophylaxis or as such treat the existing diseases or condition (e.g. bacterial infection or fungal infection). Treatment also includes treating, preventing development of, or alleviating to some extent, one or more of the symptoms of the diseases or condition.

In an embodiment, the present invention relates to a heparan sulfate compound of formula (I) or a stereoisomer, a tautomer, a conformer, a pharmaceutically acceptable salt or a pharmaceutically acceptable solvate thereof;

In another embodiment, the present invention relates to a heparan sulfate compound of formula (I) or a stereoisomer, a tautomer, a conformer, a pharmaceutically acceptable salt or a pharmaceutically acceptable solvate thereof,

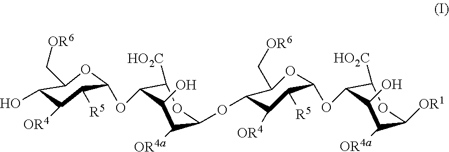

wherein:
R¹ is —(CH₂)₃NH₂;
R⁴ is H or SO₃⁻;
R^{4a} is H or SO₃⁻;
R⁵ is NH₂, or NHCOCH₃
R⁶ is H, SO₃⁻ and PO₄²⁻.

In another embodiment of the present invention, the compound of formula (I) encompasses a compound of formula (Ia) or a stereoisomer, a epimer, a conformer, a pharmaceutically acceptable salt or a pharmaceutically acceptable solvate thereof,

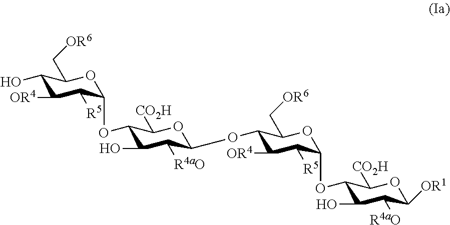

wherein:
R¹ is —(CH₂)₃NH₂;
R⁴ is H or SO₃⁻;
R^{4a} is H or SO₃⁻;
R⁵ is NHCOCH₃
R⁶ is H, or SO₃⁻.

In one aspect, the present invention relates to a heparan sulfate compound of formula (II) or a stereoisomer, a tautomer, a conformer, a pharmaceutically acceptable salt, a homooligosaccharides or a pharmaceutically acceptable solvate thereof,

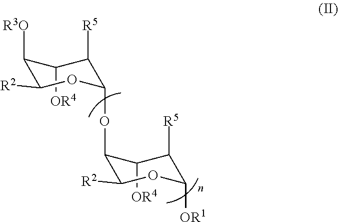

wherein:
R¹ is

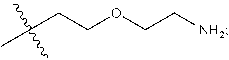 [SMILES: *CCOCCN]

R² independently represents COOH;
R³ independently represents H or SO₃⁻;
R⁴ independently represents H;
R⁵ independently represents OH, or OSO₃⁻,
n is 0, 1, 2 or 3.

In an embodiment, the compound of present invention is selected from the compounds given in the table below:

TABLE 1

| 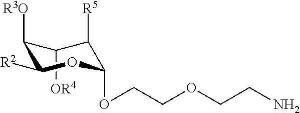 | | | | |
|---|---|---|---|---|
| Example No | R² | R³ | R⁴ | R⁵ |
| 1 | COOH | H | H | OH |
| 2 | COOH | SO₃⁻ | H | OH |
| 3 | COOH | SO₃⁻ | H | OSO₃⁻ |

TABLE 2

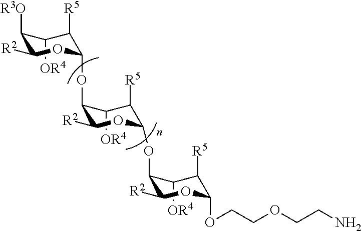

| Example No | R² | R³ | R⁴ | R⁵ | n |
|---|---|---|---|---|---|
| 4 | COOH | H | H | OH | 0 |
| 5 | COOH | SO₃⁻ | H | OH | 0 |
| 6 | COOH | SO₃⁻ | H | OSO₃⁻ | 0 |
| 7 | COOH | H | H | OH | 1 |
| 8 | COOH | SO₃⁻ | H | OH | 1 |
| 9 | COOH | SO₃⁻ | H | OSO₃⁻ | 1 |
| 10 | COOH | H | H | OH | 2 |
| 11 | COOH | SO₃⁻ | H | OH | 2 |
| 12 | COOH | SO₃⁻ | H | OSO₃⁻ | 2 |

TABLE 3

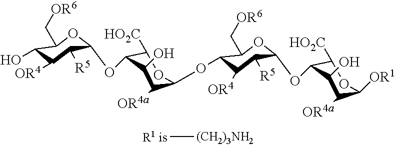

| Example No | R⁴ | R^{4a} | R⁵ | R⁶ |
|---|---|---|---|---|
| 13 | H | SO₃⁻ | NH₂ | H |
| 14 | H | SO₃⁻ | NHCOCH₃ | H |
| 15 | H | H | NH₂ | H |
| 16 | H | H | NHCOCH₃ | H |
| 17 | SO₃⁻ | H | NH₂ | SO₃⁻ |
| 18 | SO₃⁻ | SO₃⁻ | NH₂ | H |
| 19 | H | SO₃⁻ | NH₂ | SO₃⁻ |
| 20 | H | SO₃⁻ | NHCOCH₃ | SO₃⁻ |
| 21 | H | PO₄²⁻ | NHCOCH₃ | PO₄²⁻ |
| 22 | SO₃⁻ | H | NH₂ | H |
| 23 | H | H | NH₂ | SO₃⁻ |
| 24 | H | H | NHCOCH₃ | SO₃⁻ |
| 25 | SO₃⁻ | H | NHCOCH₃ | H |
| 26 | H | H | NHCOCH₃ | PO₄²⁻ |

TABLE 4

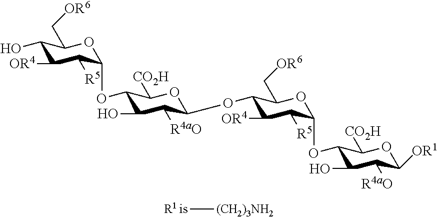

| Example No | R⁴ | R^{4a} | R⁵ | R⁶ |
|---|---|---|---|---|
| 27 | H | H | NHCOCH₃ | H |
| 28 | H | H | NHCOCH₃ | SO₃⁻ |
| 29 | SO₃⁻ | H | NHCOCH₃ | H |

TABLE 5

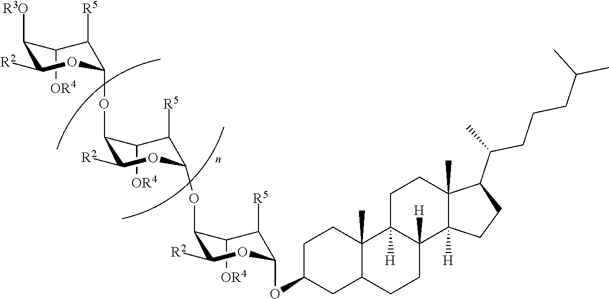

| Example No | R² | R³ | R⁴ | R⁵ | n |
|---|---|---|---|---|---|
| 30 | COOH | SO₃⁻ | H | OSO₃⁻ | 0 |
| 31 | COOH | SO₃⁻ | H | OSO₃⁻ | 1 |
| 32 | COOH | SO₃⁻ | H | OSO₃⁻ | 2 |
| 33 | CH₂OSO₃⁻ | SO₃⁻ | H | OSO₃⁻ | 0 |
| 34 | CH₂OSO₃⁻ | SO₃⁻ | H | OSO₃⁻ | 1 |
| 35 | CH₂OSO₃⁻ | SO₃⁻ | H | OSO₃⁻ | 2 |

In an embodiment, the compound of present invention is selected from the group consisting of:
Ethoxy-2-aminoethoxyl-O-α-L-idopyranoside Uronic Acid;
Ethoxy-2-aminoethoxyl-O-(4-O-sulfonato)-α-L-idopyranoside Uronic Acid;
Ethoxy-2-aminoethoxyl-O-(2,4-O-disulfonato)-α-L-idopyranoside Uronic Acid;
Ethoxy-2-aminoethoxyl-O-(α-L-idopyranosyl Uronic Acid-α(1→4))-α-L-idopyranosyl Uronic Acid;
Ethoxy-2-aminoethoxyl-O-((4-O-sulfonato)-α-L-idopyranosyl Uronic Acid-α(1→4))-α-L-idopyranoside Uronic Acid;
Ethoxy-2-aminoethoxyl-O-((2,4-O-disulfonato)-α-L-idopyranosyl Uronic Acid-α(1→4)(2-sulfonato))-α-L-idopyroside Uronic Acid;
Ethoxy-2-aminoethoxyl-O-(α-L-idopyranosyl Uronic Acid-α(1→4)-α-L-idopyranosyl Uronic Acid-α(1→4))-α-L-idopyranoside Uronic Acid;
Ethoxy-2-aminoethoxyl-O-((4-O-sulfonato)-α-L-idopyranosyl Uronic Acid-α(1→4)-L-idopyranosyl Uronic Acid-α(1→4))-α-L-idopyranoside Uronic Acid;
Ethoxy-2-aminoethoxyl-O-(2,4-O-disulfonato)-α-L-idopyranosyl Uronic Acid-α(1→4)(2-O-sulfonato)-α-L-idopyranosyl uronic acid-α(1→4)(2-O-sulfonato)-α-L-idopyranoside Uronic Acid;
Ethoxy-2-aminoethoxyl-O-(α-L-idopyranosyl Uronic Acid-α(1→4)-α-L-idopyranosyl uronic acid-α(1→4)-α-L-idopyranosyl uronic acid-α(1→4))-α-L-idopyranoside Uronic Acid;
Ethoxy-2-aminoethoxyl-O-(4-O-sulfonato)-α-L-idopyranosyl Uronic Acid-α(1→4)-α-L-Idopyranosyl Uronic Acid-α(1→4)-α-L-idopyranosyl Uronic Acid-α(1→4)-α-L-idopyranoside Uronic Acid;
Ethoxy-2-aminoethoxyl-O-((2-4-O-disulfonato)-α-L-idopyranosyl Uronic Acid-α(1->4)(2-O-sulfonato)-α-L-idopyranosyl Uronic Acid-α(1→4)(2-O-sulfonato)-α-L-idopyranosyl Uronic Acid-α(1→4)(2-O-sulfonato))-α-L-idopyranoside Uronic Acid;
3-Aminopropyl-O-[(2-amino-2-deoxy-α-D-glucopyranosyl)-(1→4)-O-(2-O-sulfonato-α-L-idopyranosyluronate)-(1→4)-O-(2-amino-2-deoxy-α-D-glucopyranosyl)]-(1→4)-O-2-O-sulfonato-α-L-idopyranosiduronate;
3-Aminopropyl-O-[(2-acetamido-2-deoxy-α-D-glucopyranosyl)-(1→4)-O-(2-O-sulfonato-α-L-idopyranosyluronate)-(1→4)-O-(2-acetamido-2-deoxy-α-D-glucopyranosyl)]-(1→4)-O-2-O-sulfonato-α-L-idopyranosiduronate;
3-Aminopropyl-O-[(2-amino-2-deoxy-α-D-glucopyranosyl)-(1→4)-O-(α-L-idopyranosyluronate)-(1→4)-O-(2-amino-2-deoxy-α-D-glucopyranosyl)]-(1→4)-O-α-L-idopyranosiduronate;
3-Aminopropyl-O-[(2-acetamido-2-deoxy-α-D-glucopyranosyl)-(1→4)-O-(α-L-idopyranosyluronate)-(1→4)-O-(2-acetamido-2-deoxy-α-D-glucopyranosyl)]-(1→4)-O-α-L-idopyranosiduronate;
3-Aminopropyl-O-[(2-amino-6-O-sulfonato-3-O-sulfonato-2-deoxy-α-D-glucopyranosyl)-(1→4)-O-(α-L-idopyranosyluronate)-(1→4)-O-(2-amino-6-O-sulfonato-3-O-sulfonato-2-deoxy-α-D-glucopyranosyl)]-(1→4)-O-α-L-idopyranosiduronate;
3-Aminopropyl-O-[(2-amino-3-O-sulfonato-2-deoxy-α-D-glucopyranosyl)-(1→4)-O-(2-O-sulfonato-α-L-idopyranosyluronate)-(1→4)-O-(2-amino-3-O-sulfonato-2-deoxy-α-D-glucopyranosyl)]-(1→4)-O-2-O-sulfonato-α-L-idopyranosiduronate;
3-Aminopropyl-O-[(2-amino-6-O-sulfonato-2-deoxy-α-D-glucopyranosyl)-(1→4)-O-(2-O-sulfonato-α-L-idopyranosyluronate)-(1→4)-O-(2-amino-6-O-sulfonato-2-deoxy-α-D-glucopyranosyl)]-(1→4)-O-2-O-sulfonato-α-L-idopyranosiduronate;
3-Aminopropyl-O-[(2-acetamido-6-O-sulfonato-2-deoxy-α-D-glucopyranosyl)-(1→4)-O-(2-O-sulfonato-α-L-idopyranosyluronate)-(1→4)-O-(2-acetamido-6-O-sulfonato-2-deoxy-α-D-glucopyranosyl)]-(1→4)-O-2-O-sulfonato-α-L-idopyranosiduronate;
3-Aminopropyl-O-[(2-acetamido-6-O-phosphonato-2-deoxy-α-D-glucopyranosyl)-(1→4)-O-(2-O-phosphonato-α-L-idopyranosyluronate)-(1→4)-O-(2-acetamido-6-O-phosphonato-2-deoxy-α-D-glucopyranosyl)]-(1→4)-O-2-O-phosphonato-α-L-idopyranosiduronate;
3-Aminopropyl-O-[(2-amino-3-O-sulfonato-2-deoxy-α-D-glucopyranosyl)-(1→4)-O-(α-L-idopyranosyluronate)-(1→4)-O-(2-amino-3-O-sulfonato-2-deoxy-α-D-glucopyranosyl)]-(1→4)-O-α-L-idopyranosiduronate;
3-Aminopropyl-O-[(2-amino-6-O-sulfonato-2-deoxy-α-D-glucopyranosyl)-(1→4)-O-(α-L-idopyranosyluronate)-(1→4)-O-(2-amino-6-O-sulfonato-2-deoxy-α-D-glucopyranosyl)]-(1→4)-O-α-L-idopyranosiduronate;
3-Aminopropyl-O-[(2-acetamido-6-O-sulfonato-2-deoxy-α-D-glucopyranosyl)-(1→4)-O-(α-L-idopyranosyluronate)-(1→4)-O-(2-acetamido-6-O-sulfonato-2-deoxy-α-D-glucopyranosyl)]-(1→4)-O-α-L-idopyranosiduronate;
3-Aminopropyl-O-[(2-acetamido-3-O-sulfonato-2-deoxy-α-D-glucopyranosyl)-(1→4)-O-(α-L-idopyranosyluronate)-(1→4)-O-(2-acetamido-3-O-sulfonato-2-deoxy-α-D-glucopyranosyl)]-(1→4)-O-α-L-idopyranosiduronate;
3-Aminopropyl-O-[(2-acetamido-6-O-phosphonato-2-deoxy-α-D-glucopyranosyl)-(1→4)-O-(α-L-idopyranosyluronate)-(1→4)-O-(2-acetamido-6-O-phosphonato-2-deoxy-α-D-glucopyranosyl)]-(1→4)-O-(α-L-idopyranosyluronate)];
3-Aminopropyl-O-[(2-deoxy-2-acetamido-α-D-glucopyranosyl)-(1→4)-O-(β-D-glucopyranosyluronate)-(1→4)-O-(2-deoxy-2-acetamido-α-D-glucopyranosyl)-(1→4)-O-(β-D-glucopyranosyluronate)];
3-Aminopropyl-O-[(2-deoxy-2-acetamido-6-O-sulfonate-α-D-glucopyranosyl)-(1→4)-O-(β-D-glucopyranosyluronate)-(1→4)-O-(2-deoxy-2-acetamido-6-O-sulfo-α-D-glucopyranosyl)-(1→4)-O-(β-D-glucopyranosyluronate)];
3-Aminopropyl-O-[(2-deoxy-2-acetamido-3-O-sulfonate-α-D-glucopyranosyl)-(1→4)-O-(β-D-glucopyranosyluronate)-(1→4)-O-(2-deoxy-2-acetamido-3-O-sulfo-α-D-glucopyranosyl)-(1→4)-O-(β-D-glucopyranosyluronate)];
Cholesteryl-O-((2,4-O-disulfonato)-α-L-idopyranosyl uronic acide-α(1→4)(2-O-sulfonato))-α-L-idopyranoside uronic acid;
Cholesteryl-O-((2,4-O-disulfonato-3-O-benzyl-)-α-L-idopyranosyl uronate-α(1→4)(2-O-sulfonato-3-O-benzyl)-α-L-idopyranosyl uronate-α(1→4)(2-O-sulfonato-3-O-benzyl))-α-L-idopyranoside urinate;
Cholesteryl-O-((2,4-O-disulfonato)-α-L-idopyranosyl uronate-α(1→4)(2-O-sulfonato)-α-L-idopyranosyl uronate-α(1→4)(2-Osulfonato)-α-L-idopyranosyl uronate-α(1→4)(2-Osulfonato))-α-L-idopyranoside urinate;
Cholestanyl-O-((2,4,6-O-trisulfonato)-α-L-idopyranosyl-α(1→4)(2,6-O-disulfonato))-α-L-idopyranoside;
Cholestanyl-O-((2,4,6-O-trisulfonato)-α-L-idopyranosyl-α(1→4)(2,6-O-disulfonato)-α-L-idopyranosyl-α(1→4)(2,6-O-disulfonato))-α-L-idopyranoside; and
Cholesteryl-O-((2,4,6-O-trisulfonato)-α-L-idopyrnosyl-α(1→4)(2,6-O-disulfonato-3-O-benzyl 3)-α-L-idopyrnosyl-α(1→4)(2,6-O-disulfonato)-α-L-idopyrnosyl-α(1→4)(2,6-O-disulfonato))-α-L-idopyranose.

The compounds of the present invention are highly potential to show anti-inflammatory activity, anti-cancer activity and neuro-protective activity.

In another embodiment, the compounds of present invention are highly potential chemokine inhibitors and are useful in the treatment of cancer diseases.

In another embodiment, the compound of formula (I) can be converted into a pharmaceutically acceptable salt. The pharmaceutical acceptable salts of the compound of formula (I) according to the invention are prepared in a manner known to one skilled in the art. Pharmaceutically acceptable salts of the compound of the present invention include but not limited to, an acid salt of a compound of the present invention containing an amine or other basic group can be obtained by reacting the compound with a suitable organic or inorganic acid, resulting in pharmaceutically acceptable anionic salt forms. Examples of anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate, and triethiodide salts.

In yet another embodiment, the pharmaceutically acceptable salts of the compound of the present invention containing acidic functional group can be prepared by reacting with a suitable base. Such a pharmaceutically acceptable salt may be made with a base which affords a pharmaceutically acceptable cation, which includes alkali metal salts (especially sodium and potassium), alkaline earth metal salts (especially calcium and magnesium), aluminum salts and ammonium salts, as well as salts made from physiologically acceptable organic bases such as trimethylamine, triethylamine, morpholine, pyridine, piperidine, picoline, dicyclohexylamine, N,N′-dibenzylethylenediamine, 2-hydroxyethylamine, bis-(2-hydroxyethyl)amine, tri-(2-hydroxyethyl)amine, procaine, dibenzylpiperidine, dehydroabietylamine, N,N′-bisdehydroabietylamine, glucamine, N-methylglucamine, collidine, quinine, quinoline, and basic amino acids such as lysine and arginine.

In another aspect, the present invention relates to pharmaceutical compositions that contain a therapeutically effective amount of a compound of formula (I) or its pharmaceutically acceptable salt in addition to customary pharmaceutically acceptable excipients. The present invention also relates to a process for the production of the pharmaceutical composition, which includes bringing a compound of formula (I), into a suitable administration form using a pharmaceutically acceptable excipient or a carrier and, if appropriate, further suitable a pharmaceutically acceptable carriers, additives or auxiliaries. The pharmaceutical compositions containing the compound of formula (I) according to the invention are prepared in a manner known to one skilled in the art.

In an embodiment, the pharmaceutical compositions can be administered orally, for example in the form of pills, tablets, coated tablets, capsules, granules or elixirs. Administration, however, can also be carried out rectally, for example in the form of suppositories, or parenterally, for example intravenously, intramuscularly or subcutaneously, in the form of injectable sterile solutions or suspensions, or topically, for example in the form of ointments or creams or transdermally, in the form of patches, or in other ways, for example in the form of aerosols or nasal sprays.

For the production of oral dosages form of the compound of formula (I) such as the pills, tablets, coated tablets and hard gelatin capsules, it is possible to use, for example, lactose, corn starch or compounds thereof, gum arabica, magnesia or glucose, etc. Pharmaceutically acceptable excipients that can be used for soft gelatin capsules and suppositories are, for example, fats, waxes, natural or hardened oils, etc. Suitable pharmaceutically acceptable excipients for the production of solutions, for example injection solutions, or of emulsions or syrups are, for example, water, physiological sodium chloride solution or alcohols, for example, ethanol, propanol or glycerol, sugar solutions, such as glucose solutions or mannitol solutions, or a mixture of the said solvents.

In another embodiment, the pharmaceutical compositions normally contain about 1% to 99%, for example, about 5% to 70%, or from about 10% to about 30% by weight of the compound of formula (I) or its pharmaceutically acceptable salt. The amount of the compound of formula (I) or its pharmaceutically acceptable salt in the pharmaceutical compositions normally is from about 5 to 500 mg or may be lower than or higher than the lower and the upper limit respectively. The dose of the compound of formula (I), which is to be administered, can cover a wide range depending on the type of disease or disorder to be treated. The dose to be administered daily is to be selected to suit the desired effect. A suitable dosage is about 0.01 to 100 mg/kg of the compound of formula (I) or its pharmaceutically acceptable salt depending on the body weight of the recipient (subject) per day, for example, about 0.1 to 50 mg/kg/day of a compound of formula (I) or a pharmaceutically acceptable salt of the compound. If required, higher or lower daily doses can also be administered.

The selected dosage level will depend upon a variety of factors including the activity of a compound of the present invention, or its salt employed, the route of administration, the time of administration, the rate of excretion of the particular compound being administered, the duration of the treatment, other concurrently administered drugs, compounds and/or materials, the age, sex, weight, condition, general health and prior medical history of the patient (subject) being treated, and like factors well known in the medical arts.

In addition to the compound of formula (I) or its pharmaceutically acceptable salt and the pharmaceutically acceptable carrier substances, the pharmaceutical compositions of the present invention can contain additives such as, for example, fillers, antioxidants, dispersants, emulsifiers, defoamers, flavors, preservatives, solubilizers or colorants. Furthermore, in addition to a compound of formula (I) or its pharmaceutically acceptable salt, the pharmaceutical compositions can also contain one or more other therapeutically or prophylactically active agents.

The present invention also encompasses within its scope the use of a compound of formula (I) or its pharmaceutically acceptable salt in combination, with other therapeutically active agents.

In an embodiment, the combination of compound of present invention with another therapeutic agent or treatment includes co-administration of a compound of formula (I) with the other therapeutic agent or treatment as either a single combination dosage form or as multiple, separate dosage forms, administration of the compound of the present invention first, followed by the other therapeutic agent or treatment and administration of the other therapeutic agent or treatment first, followed by the compound of present invention. Further therapeutic agents are administered either simultaneously or sequentially.

In another embodiment of the present invention, the other therapeutic agent may be any agent that is known in the art to treat, prevent, or reduce the symptoms of a disease or disorder. The selection of other therapeutic agent(s) is based upon the particular disease or disorder being treated. Such choice is within the knowledge of a treating physician. Furthermore, the additional therapeutic agent may be any agent when administered in combination with the administration of a compound of the present invention provides benefit to the subject in need thereof.

In another aspect, the present invention relates to a method of treatment of anti-inflammatory diseases or disorders, or cancer diseases comprising administering a therapeutically effective amount of the compound of formula (I) or a pharmaceutically acceptable salt thereof to a subject in need thereof.

While the foregoing describes various embodiments of the disclosure, other and further embodiments of the disclosure may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

The present invention is further explained in the form of following examples. However, it is to be understood that the following examples are merely illustrative and are not to be taken as limitations upon the scope of the invention. The examples of the present invention are tabulated below.

General procedure for Lev deprotection: Compound was dissolved in a mixture of dry DCM/MeOH (4/1) and 5 eq of hydrazine hydrate (H₂NNH₂·H₂O), acetic acid (for 1 g, 1 mL) was added, under nitrogen atmosphere. Allowed the reaction flask stirred for another 4 h. After completion of reaction, quenched with 10 mL of acetone, solvent was evaporated under reduced pressure. The residue was purified by flash column chromatography (EtOAc/Hexane=1/1) to afford Lev deprotected compound.

General procedure for esters deprotection: Compound was dissolved in THF/MeOH/H₂O water mixture (4/2/1) and 50 eq. of LiOH·H₂O was added. Allowed the reaction flask stirred for 2-3 days. After completion of reaction, quenched with amberlite IR120 acidic resin (If the compound is sulfated quench with Dowex 50WX8 H⁺ resin), and filtered the reaction mixture. The solvent was evaporated under reduced pressure, purified using silica column chromatography using DCM and MeOH as eluent to deprotected compounds.

General procedure for O-sulfation: Compound was dissolved in dry DMF (6 mL) and SO₃·Et₃N (OH 5 eq per OH group) was added. Allowed the reaction flask stirred for 3 days at 60° C. After completion of reaction, cooled to room temperature and added the aqueous solution of NaHCO₃ (10 eq per OH group) and kept it for another 16 hrs. Filtered the reaction mixture using whatman filter and washed with DCM/MeOH (1/1, 10 mL), solvents were evaporated under reduced pressure and the resulting residue was purified using silica column chromatography (DCM/MeOH=1/9 for mono sulfated compound). Heavily sulfated compounds were purified using Sephadex LH-20 resin, eluted with 50% of DCM/MeOH, and passed through sodium (Na⁺) resin column using water as eluent. The product fraction was lyophilized to afford sulfated compounds as a white powder.

General procedure hydrogenolysis: Compound was dissolved in dry methanol, 20% Pd(OH)₂ on carbon (0.025 g per one benzyl group) and purged with a hydrogen gas. The reaction mixture was stirred at room temperature for 2-3 days. The mixture was filtered through celite, and the filtrate was evaporated under reduced pressure. The residue was purified through bond elute C-18 column eluted with water. Sulfated compound passed through sodium (Na⁺) resin. The product fraction was lyophilized to afford sulfated compounds as a white powder.

Example 1: Synthesis of Ethaoxy-2-aminoethoxyl-O-α-L-idopyranoside Uronic Acid

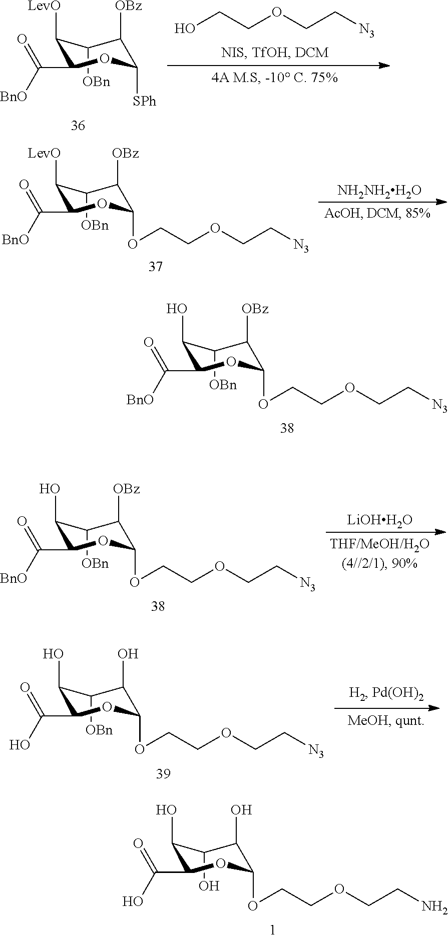

### Step-1:

Compound 36 (1.2 g, 1.79 mmol), 2-(2-azidoethoxy) ethan-1-ol (0.28 g, 2.16 mmol) and freshly dried 4 Å molecular sieves were dissolved in dry DCM (20 mL) and stirred at RT for 1 h. Then N-iodosuccinimide (0.61 g, 2.69 mmol), TfOH (0.032 mL, 0.36 mmol) were added at −10° C. and stirred for 30 min. After completion of the reaction, the reaction mixture was quenched with triethylamine and filtered through celite. The organic layer was washed with Na₂S₂O₃ followed by NaHCO₃, brine solution and dried over Na₂SO₄, then filtered and concentrated under reduced pressure. The residue obtained was purified by column chromatography (EtOAc/Hexane=1/4) to afford compound 37 (0.94 g, 75%) as syrup. ¹H NMR (400 MHz, Chloroform-d) δ 8.07-8.04 (m, 2H), 7.59-7.54 (m, 1H), 7.44-7.27 (m, 12H), 5.30-5.27 (m, 2H), 5.19 (d, J=1.2 Hz, 2H), 5.16 (d, J=11.9 Hz, 1H), 5.05 (d, J=2.2 Hz, 1H), 4.83 (d, J=11.6 Hz, 1H), 4.72 (d, J=11.6 Hz, 1H), 3.98-3.92 (m, 1H), 3.88 (dt, J=3.2, 1.7 Hz, 1H), 3.76-3.6 (m, 3H), 3.62-3.52 (m, 2H), 3.17 (t, J=5.0 Hz, 2H), 2.48 (td, J=6.7, 2.6 Hz, 2H), 2.31 (ddd, J=17.1, 7.5, 6.4 Hz, 1H), 2.20 (dt, J=17.2, 6.5 Hz, 1H), 2.06 (s, 3H). ¹³C NMR (101 MHz, CHLOROFORM-D) δ 205.98, 171.66, 168.51, 165.32, 137.65, 135.42, 133.67, 129.97, 129.48, 129.07, 128.70, 128.62, 128.55, 128.45, 127.94, 127.80, 98.61, 72.71, 72.46, 70.34, 70.22, 68.40, 68.08, 67.34, 66.93, 66.09, 50.82, 37.80, 29.73, 27.90. HRMS m/z calculated for C₃₆H₃₉O₁₁N₃Na: 712.2482; found: 712.2481.

Step-2: Benzyl(2-O-benzoyl-3-O-benzyl-4-hydroxyl)-α-L-idopyranosyl)ethoxy)-2-azidoethoxyl)carboxylate

Compound 37 (0.9 g, 1.29 mmol) was dissolved in a mixture of dry DCM/MeOH (4/1, 10 mL) and hydrazinehydrate (0.32 mL, 6.45 mmol), acetic acid (1.12 mL, 19.59 mmol) was added, under nitrogen atmosphere. The reaction flask was stirred for another 4 h. After completion of the reaction, quenched with 5 mL of acetone, solvents were evaporated under reduced pressure. The residue was purified by flash column chromatography (EtOAc/Hexane=1/1) to afford compound 38 (1.9 g, 80%) as syrup. ¹H NMR (400 MHz, Chloroform-d) δ 8.00 (dd, J=8.3, 1.4 Hz, 2H), 7.62-7.57 (m, 1H), 7.48-7.43 (m, 2H), 7.40-7.28 (m, 10H), 5.32 (d, J=12.3 Hz, 1H), 5.27-5.26 (m, 1H), 5.24 (d, J=12.3 Hz, 1H), 5.17 (s, 1H), 5.02 (d, J=1.7 Hz, 1H), 4.84 (d, J=11.6 Hz, 1H), 4.65 (d, J=11.6 Hz, 1H), 4.16-4.12 (m, 1H), 3.98-3.93 (m, 1H), 3.89 (td, J=3.0, 1.3 Hz, 1H), 3.76-3.67 (m, 3H), 3.63-3.53 (m, 2H), 3.19 (t, J=5.0 Hz, 2H), 2.80 (d, J=11.6 Hz, 1H). ¹³C NMR (101 MHz, Chloroform-d) δ 169.54, 165.11, 137.70, 135.49, 133.84, 129.89, 129.14, 128.77, 128.73, 128.55, 128.52, 128.44, 128.02, 127.88, 99.00, 74.75, 72.16, 70.33, 70.25, 68.48, 68.29, 67.96, 67.47, 67.17, 50.83. HRMS m/z calculated for C₃₁H₃₃N₃O₉Na: 614.2114 found: 614.2109.

Step-3: (3-O-Benzyl-2,4-dihydroxyl)-α-L-idopyranosyl)ethoxy)-2-azidoethoxyl)carboxylic acid

Followed general procedure for ester deprotection of compound 38 which yielded compound 39 (90%). ¹H NMR (400 MHz, Methanol-d4) δ 7.41-7.25 (m, 5H), 4.75-4.71 (m, 2H), 4.65-4.60 (m, 1H), 4.08 (s, 1H), 3.93-3.87 (m, 1H), 3.81-3.80 (m, 1H), 3.71-3.66 (m, 5H), 3.64-3.56 (m, 1H), 3.23 (t, J=4.9 Hz, 2H). ¹³C NMR (101 MHz, Methanol-d4) δ 173.88, 139.69, 129.32, 128.86, 128.67, 102.77, 78.04, 72.92, 71.28, 71.21, 69.92, 69.67, 69.24, 68.10, 51.75. HRMS m/z calculated for C₁₇H₂₃N₃O₉Na: 420.1383 found: 420.1378.

Step-4: Ethaoxy-2-aminoethoxyl-O-α-L-idopyranoside Uronic Acid

Followed general procedure for hydrogenolysis of compound 39 to obtain Example 1 (90%). 1H NMR (400 MHz, Deuterium Oxide) δ 4.86 (d, J=4.2 Hz, 1H), 4.62 (d, J=3.6 Hz, 1H), 3.91-3.86 (m, 2H), 3.74 (dd, J=6.9, 5.1 Hz, 2H), 3.71-3.68 (m, 4H), 3.51 (dd, J=6.4, 4.2 Hz, 1H), 3.14 (t, J=5.1 Hz, 2H). 13C NMR (101 MHz, Deuterium Oxide) δ 173.78, 101.00, 71.07, 70.15, 69.96, 69.85, 69.67, 67.92, 66.34, 39.08. HRMS m/z calculated for C₁₀H₁₉NO₈Na: 304.1008 found: 304.1001.

Example 2: Synthesis of Ethoxy-2-aminoethoxyl-O-(4-O-sulfonato)-α-L-idopyranoside Uronic Acid

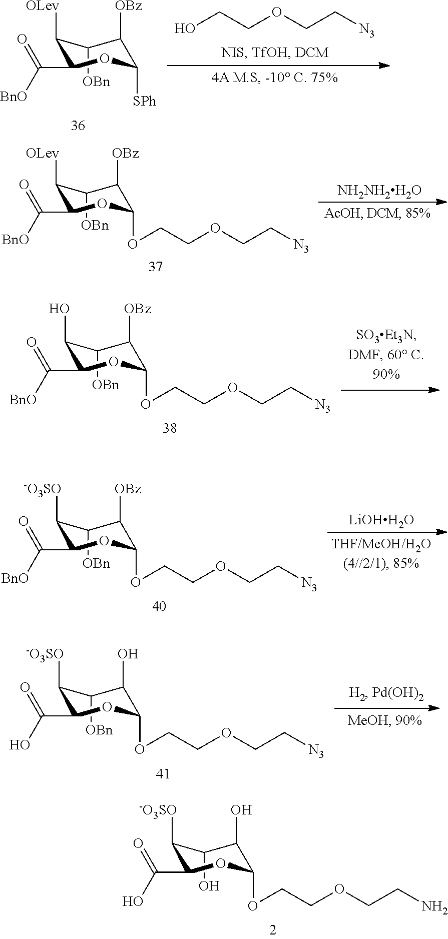

Step-1: The compound 38 was prepared by following the experimental procedure given above.
Step-2: Benzyl(2-O-benzoyl-3-O-benzyl-4-sulfo)-α-L-idopyranosyl)ethoxy) 2-azidoethoxyl)carboxylate

Followed general procedure for sulfation of compound 38 yielded compound 40 (90%). ¹H NMR (400 MHz, Methanol-d₄) δ 8.17 (dd, J=8.4, 1.3 Hz, 2H), 7.61-7.57 (m, 1H), 7.47 (ddd, J=8.6, 4.4, 2.8 Hz, 4H), 7.40-7.32 (m, 5H), 7.30-7.22 (m, 3H), 5.37 (d, J=12.0 Hz, 1H), 5.15-5.10 (m, 2H), 5.09 (d, J=2.3 Hz, 1H), 5.07 (s, 1H), 4.81-4.76 (m, 2H), 4.65 (d, J=60.6 Hz, 1H), 4.39 (ddd, J=3.5, 2.7, 1.1 Hz, 1H), 3.88-3.83 (m, 1H), 3.70-3.62 (m, 3H), 3.59-3.46 (m, 2H), 3.09 (t, J=5.1 Hz, 2H). ¹³C NMR (101 MHz, Methanol-d₄) δ 170.70, 167.13, 139.36, 136.96, 134.34, 131.33, 130.94, 129.77, 129.49, 129.46, 129.28, 128.99, 128.70, 99.71, 74.93, 73.48, 73.08, 71.27, 71.11, 69.43, 68.97, 68.51, 68.46, 51.70. HRMS m/z calculated for C₃₁H₃₃N₃O₁₂S: 670.1712 found (M−H): 669.1715.

Step-3: (2-Hydroxyl-3-O-benzyl-4-sulfo)-α-L-idopyranosyl)ethoxy) 2-azidoethoxyl)carboxylic acid

Followed general procedure for deprotection of compound 40 yielded compound 41 (85%). ¹H NMR (400 MHz, Deuterium Oxide) δ 7.52-7.41 (m, 5H), 4.92 (dd, J=2.0, 0.9 Hz, 1H), 4.83 (d, J=11.7 Hz, 1H), 4.70 (d, J=11.6 Hz, 1H), 4.61 (d, J=2.3 Hz, 1H), 4.20 (td, J=3.3, 1.0 Hz, 1H), 3.93-3.87 (m, 1H), 3.76-3.68 (m, 6H), 3.44-3.42 (m, 2H). ¹³C NMR (101 MHz, Deuterium Oxide) δ 174.75, 137.21, 128.63, 128.62, 128.32, 100.31, 74.72, 73.42, 72.37, 69.48, 69.30, 67.75, 67.47, 67.38, 50.13. HRMS m/z calculated for C₁₇H₂₂N₃O₁₁S⁻: 476.0981 found (M−H): 475.0980.

Step-4: Ethoxy-2-aminoethoxyl-O-(4-O-sulfonato)-α-L-idopyranoside Uronic Acid

Followed general procedure for hydrogenolysis of compound 41 to obtain Example 2 (90%). 1H NMR (400 MHz, Deuterium Oxide) δ 4.78 (d, J=3.5 Hz, 1H), 4.46 (d, J=3.1 Hz, 1H), 4.41-4.39 (m, 1H), 4.11 (t, J=5.0 Hz, 1H), 3.80-3.75 (m, 1H), 3.67 (dt, J=11.8, 4.2 Hz, 1H), 3.62-3.59 (m, 4H), 3.47-3.45 (m, 1H), 3.07-3.04 (m, 2H). ¹³C NMR (101 MHz, Deuterium Oxide) δ 174.64, 100.66, 76.67, 69.84, 69.66, 69.00, 68.92, 67.79, 66.29, 39.10. HRMS m/z calculated for C₁₀H₁₈NO₁₁S⁻: 360.0606 found: 360.0611.

Examples 3-12: The following examples 3-12 were synthesized by following the above experimental procedures with appropriate starting materials and non-critical variations.

| Ex. | Structure and Name | NMR & Mass |
|---|---|---|
| 3 | 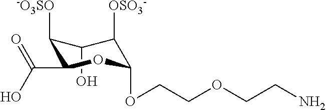 Ethoxy-2-aminoethoxyl-O-(2,4-0- disulfonato)--l-idopyranoside Uronic Acid | ¹H NMR (400 MHz, Deuterium Oxide) δ 5.14 (s, 1H), 4.59 (d, J = 2.0 Hz, 1H), 4.57 (d, J = 2.8 Hz, 1H), 4.48 (dt, J = 2.8, 1.4 Hz, 1H), 4.21 (dt, J = 2.5, 1.2 Hz, 1H), 3.84 (ddd, J = 13.9, 8.6, 4.4 Hz, 2H), 3.73 (q, J = 4.7, 4.2 Hz, 4H), 3.20-3.18 (m, 2H). ¹³C NMR (101 MHz, Deuterium Oxide) δ 174.88, 98.67, 74.60, 73.28, 69.86, 67.51, 66.46, 66.37, 66.26, 39.18. HRMS m/z calculated for C₁₀H₂₇NO₁₄S₂²⁻: 219.5051 found: 277.5059. |
| 4 | 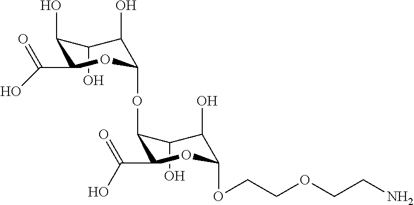 | ¹H NMR (400 MHz, Deuterium Oxide) δ 4.95 (d, J = 3.0 Hz, 1H), 4.89 (d, J = 3.4 Hz, 1H), 4.57 (d, J = 3.2 Hz, 1H), 4.55 (d, J = 2.7 Hz, 1H), 4.10-4.09 (m, 1H), 3.96-3.90 (m, 3H), 3.83- 3.75 (m, 6H), 3.63-3.60 (m, 2H), 3.23- 3.20 (m, 2H). ¹³C NMR (101 MHz, Deuterium Oxide) δ 176.14, 175.60, 102.36, 101.09, 78.52, 70.55, 70.40, 70.04, 69.86, 69.80, 69.29, 69.13, 68.82, 67.50, 66.29, 39.08. HRMS m/z calculated for C₁₆H₂₇NO₁₄Na: 457.1432 found: 457.1443 |
| | Ethoxy-2-aminoethoxyl-O-(α-I-idopyranosyl | |
| | Uronic Acid-a(1 → 4))-α-l-idopyranosyl | |
| | Uronic Acid | |
| 5 | 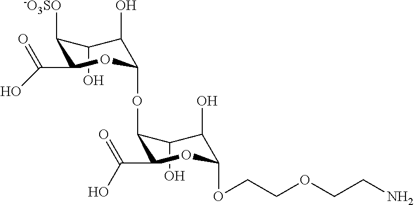 Ethoxy-2-aminoethoxyl-O-((4-O-sulfonato)- α-I-idopyranosyl Uronic Acid-α(1 → 4))-α-l-idopyranoside Uronic Acid | ¹H NMR (400 MHz, Deuterium Oxide) δ 4.87 (dd, J = 4.4, 2.7 Hz, 2H), 4.66 (d, J = 2.4 Hz, 1H), 4.52 (t, J = 3.1 Hz, 1H), 4.47 (d, J = 2.7 Hz, 1H), 4.20 (t, J = 3.8 Hz, 1H), 4.01 (dd, J = 4.2, 2.8 Hz, 1H), 3.87-3.81 (m, 2H), 3.74-3.66 (m, 6H), 3.59 (ddd, J = 4.2, 2.3, 1.0 Hz, 1H), 3.53 (dd, J = 5.3, 3.0 Hz, 1H), 3.14-3.12 (m, 1H). ¹³C NMR (101 MHz, D₂O) δ 175.54, 174.70, 102.20, 101.13, 78.41, 76.12, 69.80, 69.12, 68.88, 68.30, 68.06, 67.92, 67.49, 66.30, 39.09. HRMS m/z calculated for C₁₆H₂₆NO₁₇S⁻: 536.0927 found: 536.0899 |
| 6 | 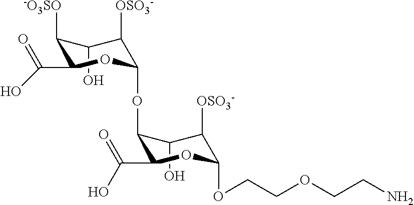 | ¹H NMR (600 MHz, Deuterium Oxide) δ 5.05 (s, 2H), 4.76 (s, 1H), 4.54 (s, 1H), 4.47 (s, 1H), 4.40 (s, 1H), 4.13 (s, 1H), 4.09 (s, 1H), 3.96 (d, J = 8.8 Hz, 2H), 3.78-3.70 (m, 3H), 3.65-3.62 (m, 3H), 3.09 (t, J = 5.1 Hz, 2H). ¹³C NMR (151 MHz, D₂O) δ 174.96, 174.07, 100.74, 98.94, 77.93, 74.24, 74.03, 71.88, 69.80, 68.04, 67.61, 67.39, 66.21, 65.53, 39.07. HRMS m/z calculated for C₁₆H₂₄NO₂₄S₃₃⁻: 231.3306 found: 231.3313. |
| | Ethoxy-2-aminoethoxyl-O-((2,4-O- | |
| | disulfonato)-α-l-idopyranosyl Uronic Acid- | |
| | α(1 → 4)(2-sulfonato))-α-L-idopyroside | |
| | Uronic Acid | |
| 7 | 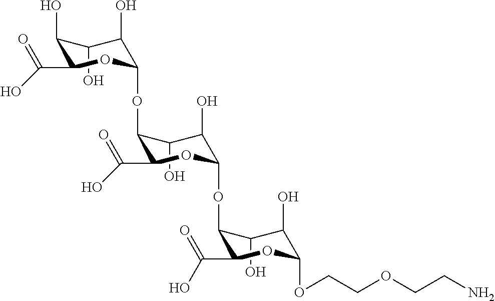 | ¹H NMR (400 MHz, Deuterium Oxide) δ 4.95 (d, J = 2.4 Hz, 2H), 4.90 (d, J = 3.1 Hz, 1H), 4.67 (d, J = 2.1 Hz, 1H), 4.57 (d, J = 2.8 Hz, 1H), 4.55 (d, J = 2.4 Hz, 1H), 4.11 (dt, J = 11.6, 3.1 Hz, 2H), 3.94 (ddd, J = 15.7, 8.6, 4.2 Hz, 4H), 3.82 (q, J = 4.3 Hz, 2H), 3.78- 3.74 (m, 4H), 3.63 (dd, J = 8.0, 4.8 Hz, 3H), 3.21 (t, J = 5.1 Hz, 2H). ¹³C NMR (101 MHz, D₂O) δ 176.11, 175.61, 175.55, 102.50, 102.41, 101.09, 77.97, 77.87, 70.23, 70.18, 70.15, 69.82, 69.50, 68.88, 68.52, 68.35, 68.13, 67.47, 66.27, 39.08. HRMS m/z calculated for C₂₂H₃₅NO₂₀Na: 656.1650 found: 656.1659. |
| | Ethoxy-2-aminoethoxyl-O-(α-L-idopyranosyl | |
| | Uronic Acid-a(1 → 4)-α-L-idopyranosyl | |
| | Uronic Acid-a(1 → 4))-α-L-idopyranoside | |
| | Uronic Acid | |
| 8 | 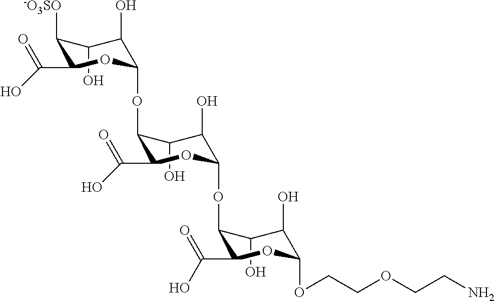 | ¹H NMR (400 MHz, Deuterium Oxide) δ 4.87 (dd, J = 5.6, 2.3 Hz, 3H), 4.67 (d, J = 2.4 Hz, 1H), 4.59 (d, J = 2.3 Hz, 1H), 4.51 (t, J = 3.0 Hz, 1H), 4.47 (d, J = 2.4 Hz, 1H), 4.19 (td, J = 3.8, 1.0 Hz, 1H), 4.02 (dt, J = 10.4, 3.2 Hz, 2H), 3.82 (q, J = 5.0 Hz, 2H), 3.74-3.65 (m, 5H), 3.58 (ddd, J = 3.9, 2.2, 1.0 Hz, 1H), 3.55-3.51 (m, 2H), 3.15-3.09 (m, 1H). ¹³C NMR (101 MHz, D₂O) δ 175.51, 175.38, 174.38, 102.56, 102.29, 101.10, 78.00, 77.89, 75.84, 69.81, 69.53, 68.88, 68.53, 68.45, 68.18, 68.04, 67.82, 67.65, 67.48, 66.28, 39.08. HRMS m/z calculated for C₂₂H₃₄NO₂₃S⁻: 712.1248 found: 712.1229. |
| | Ethoxy-2-aminoethoxyl-O-((4-O-sulfonato)- | |
| | α-L-idopyranosyl Uronic Acid-a(1 → 4)-L- | |
| | idopyranosyl Uronic Acid-a(1 → 4))-α-L- | |
| | idopyranoside Uronic Acid | |
| 9 | 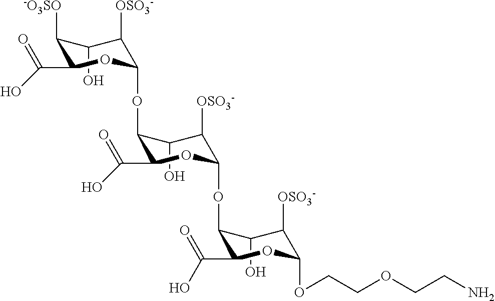 | ¹H NMR (600 MHz, Deuterium Oxide) δ 5.19 (s, 1H), 5.17-5.16 (m, 2H), 4.68 (t, J = 2.3 Hz, 2H), 4.57 (d, J = 2.1 Hz, 1H), 4.52 (td, J = 2.6, 1.2 Hz, 1H), 4.27 (dt, J = 2.4, 1.2 Hz, 1H), 4.25 (dd, J = 2.6, 1.3 Hz, 1H), 4.21 (t, J = 2.9 Hz, 1H), 4.17 (t, J = 2.4 Hz, 1H), 4.09- 4.07 (m, 3H), 3.90-3.82 (m, 1H), 3.80- 3.72 (m, 5H), 3.22-3.21 (m, 2H). ¹³C NMR (151 MHz, D₂O) δ 175.13, 174.92, 174.36, 100.79, 99.00, 77.75, 77.01, 74.33, 72.22, 71.67, 69.89, 68.14, 67.71, 67.47, 67.31, 67.14, 66.53, 66.29, 65.56, 39.16. HRMS m/z calculated for C₂₂H₃₁NO₃₂S₄⁴⁻: 237.2433 found: 237.2439. |
| | Ethoxy-2-aminoethoxyl-O-(2,4-O- | |
| | disulfonato)-α-l-idopyranosyl uronic acid-α | |
| | (1 → 4)(2-O-sulfonato)-α-l-idopyranosyl uronic | |
| | acid-a(1 → 4)(2-O-sulfonato)-α-1- | |
| | idopyranoside uronic acid | |
| 10 | 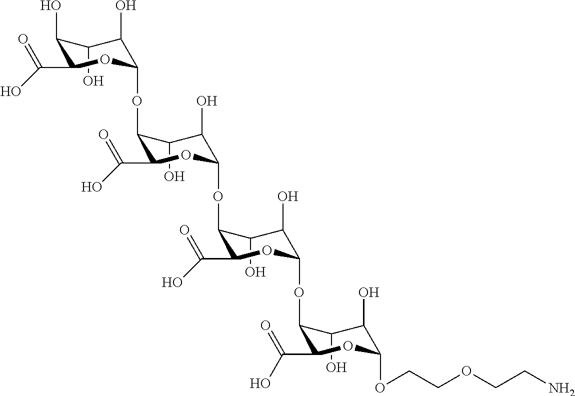 | ¹H NMR (600 MHz, Deuterium Oxide) δ 4.99-4.91 (m, 6H), 4.14 (d, J = 12.9 Hz, 3H), 4.03 (t, J = 3.8 Hz, 1H), 3.92 (dt, J = 9.8, 4.5 Hz, 3H), 3.84 (t, J = 5.2 Hz, 1H), 3.80-3.73 (m, 5H), 3.65- 3.58 (m, 5H), 3.20 (t, J = 5.0 Hz, 2H). ¹³C NMR (151 MHz, D₂O) δ 173.00, 172.78, 172.72, 102.78, 102.74, 102.64, 101.12, 77.61, 77.58, 77.50, 70.02, 69.83, 69.62, 69.47, 69.25, 69.03, 68.96, 68.84, 68.56, 68.40, 68.05, 67.94, 67.70, 39.01. HRMS m/z calculated for C₂₈H₄₃NO₂₆Na: 832.1971 found: 832.1987. |
| | Ethoxy-2-aminoethoxyl-O-(a-l-idopyranosyl | |
| | uronic acid-a(1 → 4)-α-l-idopyranosyl uronic | |
| | acid-a(1 → 4)-α-l-idopyranosyl uronic acid- | |
| | a(1 → 4))-α-l-idopyranoside uronic acid | |
| 11 | 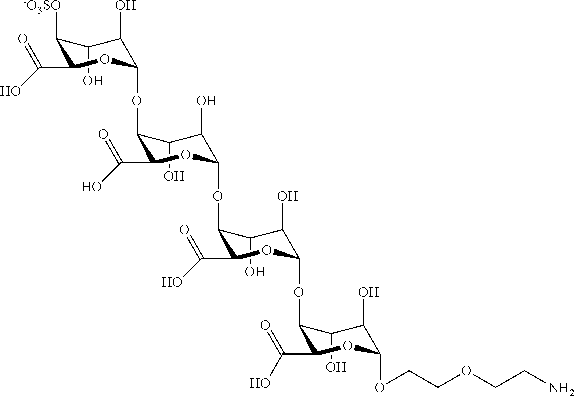 | ¹H NMR (600 MHz, Deuterium Oxide) δ 4.90 (s, 1H), 4.87-4.54 (m, 3H), 4.65 (d, J = 8.2 Hz, 2H), 4.53-4.51 (m, 2H), 4.16 (t, J = 3.7 Hz, 1H), 4.03 (d, J = 14.3 Hz, 3H), 3.82 (p, J = 4.4 Hz, 4H), 3.72-3.63 (m, 5H), 3.58 (d, J = 4.1 Hz, 1H), 3.52 (dq, J = 5.2, 2.5 Hz, 3H), 3.11 (t, J = 5.0 Hz, 2H). ¹³C NMR (151 MHz, D₂O) δ 174.83, 174.64, 174.44, 173.09, 102.68, 102.42, 102.18, 101.00, 77.87, 77.76, 77.37, 75.18, 69.66, 69.34, 68.84, 68.64, 68.42, 68.31, 68.20, 68.07, 68.00, 67.82, 67.52, 67.43, 67.22, 66.18, HRMS m/z calculated for C₂₈H₄₂NO₂₉S⁻: 888.1569 found: 888.1556. |
| | Ethoxy-2-aminoethoxyl-O-(4-O-sulfonato)-α- | |
| | 1-idopyranosyl Uronic Acid-a(1 → 4)-α-1- | |
| | Idopyranosyl Uronic Acid-a(1 → 4)-α-1- | |
| | idopyranosyl Uronic Acid-a(1 → 4)-α-l- | |
| | idopyranoside Uronic Acid | |
| 12 | 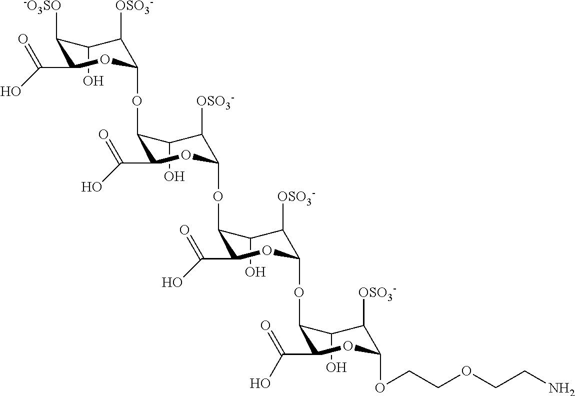 | ¹H NMR (600 MHz, Deuterium Oxide) δ 5.17-5.11 (m, 4H), 5.06 (t, J = 6.1 Hz, 3H), 4.78 (s, 1H), 4.57 (s, 1H), 4.35 (s, 1H), 4.13-4.03 (m, 10H), 3.81 (dt, J = 12.1, 4.0 Hz, 1H), 3.74 (dt, J = 12.1, 3.8 Hz, 1H), 3.67 (q, J = 7.3, 6.1 Hz, 4H), 3.12 (t, J = 4.9 Hz, 2H). ¹³C NMR (151 MHz, D₂O) δ 172.79, 172.36, 171.79, 102.17, 102.03, 101.97, 98.80, 78.81, 78.63, 78.53, 73.31, 73.15, 72.26, 69.63, 67.65, 67.03, 66.76, 66.61, 66.34, 65.57, 39.09. HRMS m/z calculated for C₂₈H₃₈NO₄₁S₅⁵⁻: 240.7910 found: 240.7912. |
| | Ethoxy-2-aminoethoxyl-O-((2-4-0- | |
| | disulfonato)-α-l-idopyranosyl Uronic Acid- | |
| | α(1 → 4)(2-O-sulfonato)-α-l-idopyranosyl | |
| | Uronic Acid-a(1 → 4)(2-O-sulfonato)-α-l- | |
| | idopyranosyl Uronic Acid-α(1 → 4)(2-0- | |
| | sulfonato))-α-l-idopyranoside Uronic Acid | |

### Synthesis of HS Tetrasaccharide Precursor Compounds

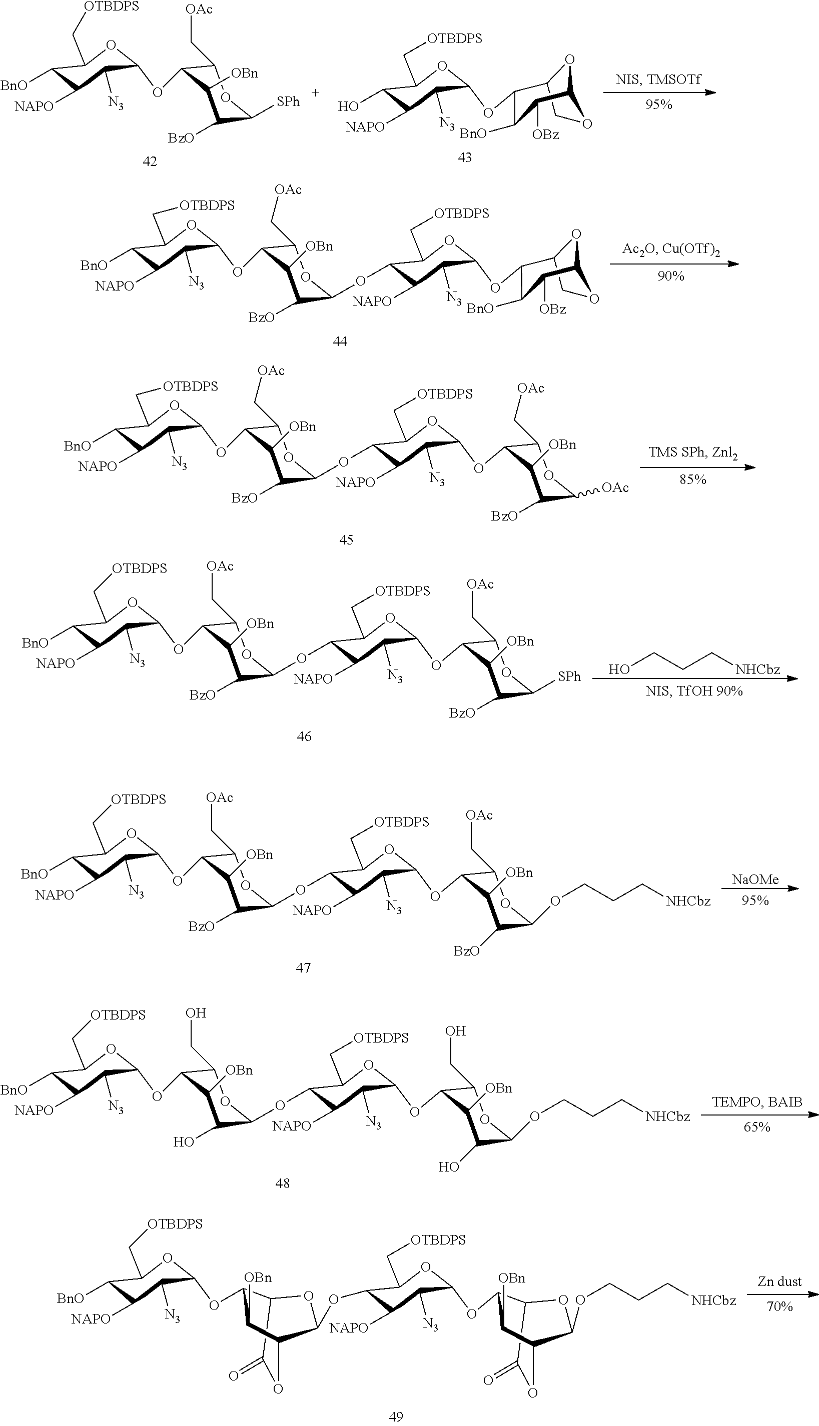

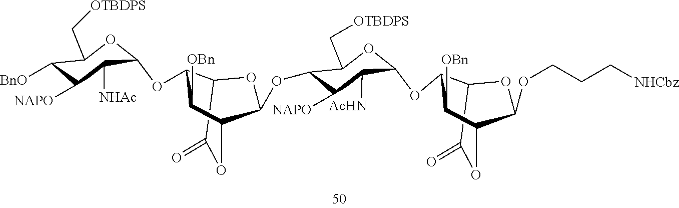

### 1,6-Anhydro-[(2-azido-4-O-benzyl-6-O-tert-butyldiphenylsilyl-3-O-(2-naphthylmethyl)-2-deoxy-α-D-glucopyranosyl)-(1→4)-O-(2-O-benzoyl-3-O-benzyl-6-O-acetyl-α-L-idopyranosyl)-(1→4)-O-(2-azido-6-O-tert-butyldiphenylsilyl-3-O-(2-naphthylmethyl)-2-deoxy-α-D-glucopyranosyl)]-(1→4)-O-2-O-benzoyl-3-O-benzyl-β-L-idopyranose (44)

A solution of compound 42 (0.87 g, 0.74 mmol) and compound 43 (0.55 g, 0.60 mmol) in CH₂Cl₂ (15 mL) was stirred under N₂ atmosphere in round bottom flask containing freshly dried 4 Å molecular sieves for 2 h. The mixture solution was cooled to −10° C. followed by the addition of NIS (0.26 g, 1.18 mmol) and TMSOTf (26 μL, 0.148 mmol). After 15 minutes, the reaction completion was monitored by TLC and quenched using few drops of Et3N. Molecular sieves were filtered using celite and the organic layer was washed with aqueous Na₂S₂O₃ and brine. The collected organic layer was dried over Na₂SO₄, filtered, concentrated and purified through silica gel column chromatography (ethyl acetate/hexane=1/6, v/v) to obtain compound 44 in 95% yield. ¹H NMR (400 MHz, Chloroform-d) δ 8.03 (dd, J=6.5, 1.4 Hz, 4H), 7.83-7.81 (m, 1H), 7.78-7.75 (m, 1H), 7.71-7.58 (m, 15H), 7.48-7.27 (m, 29H), 7.22-7.19 (m, 9H), 5.51 (d, J=1.7 Hz, 1H), 5.36 (s, 1H), 5.22 (s, 1H), 5.18 (d, J=3.9 Hz, 1H), 5.13 (d, J=11.3 Hz, 1H), 5.04 (dd, J=8.2, 1.7 Hz, 1H), 4.87 (d, J=11.3 Hz, 1H), 4.83 (d, J=4.2 Hz, 1H), 4.81-4.80 (m, 1H), 4.75 (s, 1H), 4.73-4.67 (m, 3H), 4.63 (d, J=10.6 Hz, 1H), 4.50 (dt, J=6.8, 3.5 Hz, 1H), 4.41-4.38 (m, 1H), 4.36 (d, J=4.78 Hz, 1H), 4.18-4.06 (m, 4H), 4.01 (tt, J=7.0, 3.9 Hz, 2H), 3.93-3.86 (m, 4H), 3.83 (dd, J=9.4, 2.9 Hz, 2H), 3.74 (t, J=9.8 Hz, 3H), 3.66-3.61 (m, 2H), 3.59-3.51 (m, 2H), 3.38 (dd, J=10.4, 3.8 Hz, 1H), 3.31 (dd, J=10.1, 3.5 Hz, 1H), 1.41 (s, 3H), 1.03 (s, 9H), 1.00 (s, 9H). ¹³C NMR (101 MHz, Chloroform-d) δ 170.07, 165.75, 138.11, 137.91, 137.44, 135.87, 135.82, 135.66, 135.59, 135.45, 135.21, 133.43, 133.37, 133.29, 133.21, 133.15, 133.03, 132.98, 132.86, 129.89, 129.82, 129.76, 129.72, 129.55, 129.43, 128.66, 128.55, 128.48, 128.44, 128.40, 128.36, 128.21, 128.15, 127.98, 127.84, 127.79, 127.76, 127.71, 127.67, 127.62, 127.53, 127.00, 126.94, 126.30, 126.08, 126.06, 125.97, 125.76, 125.72, 99.35, 99.21, 98.32, 97.35, 80.38, 79.21, 79.04, 78.45, 77.70, 77.25, 76.95, 75.35, 75.30, 75.10, 74.95, 74.13, 73.46, 73.34, 73.00, 72.95, 72.90, 72.83, 68.87, 65.52, 65.34, 64.04, 62.42, 62.36, 61.98, 26.86, 26.80, 20.20, 19.35, 19.33. HR-ESI-MS (m/z): [M+Na]⁺ calcd for C₁₁₅H₁₁₈N₆O₂₁Si₂Na, 1997.7787; found, 1997.7722.

### Acetyl-O-[(2-azido-4-O-benzyl-6-O-tert-butyldiphenylsilyl-3-O-(2-naphthylmethyl)-2-deoxy-α-D-glucopyranosyl)-(1→4)-O-(2-O-benzoyl-3-O-benzyl-6-O-acetyl-α-L-idopyranosyl)-(1→4)-O-(2-azido-6-O-tert-butyldiphenylsilyl-3-O-(2-naphthylmethyl)-2-deoxy-α-D-glucopyranosyl)]-(1→4)-O-2-O-benzoyl-3-O-benzyl-6-O-acetyl-α/β-L-idopyranoside (45)

A solution of compound 44 (1 g, 0.98 mmol) in Ac₂O (10 mL) was stirred at ice cold temperature for 15 minutes before the addition of Cu(OTf)₂ (0.035 g, 0.098 mmol). After 16 h, the reaction mixture was concentrated under reduced pressure and the residue was extracted with ethyl acetate, NaHCO₃ and washed with brine. The combined organic layer was dried over Na₂SO₄, filtered, concentrated and purified through silica gel column chromatography (ethyl acetate/hexane=1/5, v/v) to obtain compound 45 in 90% yield. ¹H NMR (400 MHz, Chloroform-d) δ 8.16-8.11 (m, 3H), 8.05 (d, J=7.7 Hz, 3H), 7.86-7.77 (m, 4H), 7.64 (ddq, J=22.1, 13.3, 7.6, 6.4 Hz, 21H), 7.56-7.29 (m, 50H), 7.28-7.14 (m, 17H), 6.24 (d, J=2.3 Hz, 1H), 5.42 (s, 1H), 5.25-5.16 (m, 3H), 4.95-4.67 (m, 13H), 4.61 (dd, J=7.2, 3.4 Hz, 2H), 4.41 (dq, J=16.1, 6.1, 5.4 Hz, 5H), 4.30-4.10 (m, 8H), 4.09-4.01 (m, 3H), 3.93-3.63 (m, 18H), 3.52 (s, 1H), 3.35-3.30 (m, 3H), 2.12 (d, J=3.9 Hz, 2H), 1.97 (s, 2H), 1.90 (s, 3H), 1.28 (s, 2H), 1.25 (s, 3H), 1.05 (s, 15H), 1.00 (s, 13H). ¹³C NMR (101 MHz, Chloroform-d) δ 170.44, 170.31, 169.98, 169.97, 169.04, 168.91, 166.05, 165.76, 165.71, 138.18, 137.52, 137.51, 137.24, 135.90, 135.87, 135.64, 135.60, 135.49, 135.45, 135.23, 133.46, 133.40, 133.36, 133.34, 133.28, 133.25, 133.22, 133.17, 133.16, 133.02, 132.99, 132.74, 132.71, 129.90, 129.80, 129.76, 129.72, 129.65, 129.63, 129.50, 128.72, 128.69, 128.63, 128.60, 128.52, 128.47, 128.44, 128.36, 128.23, 128.18, 128.15, 128.13, 128.09, 128.05, 128.00, 127.98, 127.84, 127.76, 127.75, 127.71, 127.66, 127.63, 127.53, 127.43, 127.41, 127.37, 126.94, 126.10, 126.04, 126.00, 125.94, 125.88, 125.86, 125.69, 125.67, 125.61, 125.58, 98.52, 98.13, 97.91, 97.00, 96.98, 91.74, 90.56, 80.36, 79.11, 78.91, 77.64, 77.27, 75.31, 75.04, 73.97, 73.88, 73.83, 73.33, 73.29, 73.26, 72.93, 72.80, 72.68, 72.55, 72.46, 71.98, 68.63, 68.54, 67.80, 67.25, 64.65, 64.37, 64.29, 64.06, 62.48, 62.33, 62.26, 61.94, 26.87, 26.79, 21.01, 20.73, 20.66, 20.01, 19.95, 19.44, 19.34; HR-ESI-MS (m/z): [M+Na]⁺ calcd for C₁₁₉H₁₂₄N₆O₂₄Si₂Na, 2100.8137; found, 2100.8171.

### Phenyl-[(2-azido-4-O-benzyl-6-O-tert-butyldiphenylsilyl-3-O-(2-naphthylmethyl)-2-deoxy-α-D-glucopyranosyl)-(1→4)-O-(2-O-benzoyl-3-O-benzyl-6-O-acetyl-α-L-idopyranosyl)-(1→4)-O-(2-azido-6-O-tert-butyldiphenylsilyl-3-O-(2-naphthylmethyl)-2-deoxy-α-D-glucopyranosyl)]-(1→4)-O-2-O-benzoyl-3-O-benzyl-6-O-acetyl-1-thio-α-L-idopyranoside (46)

A solution of compound 45 (0.98 g, 0.88 mmol), ZnI₂ (0.59 g, 1.84 mmol) and phenyl trimethylsilyl sulphide (0.50 g, 2.72 mmol) in CH₂Cl₂ (15 mL) was stirred under N₂ atmosphere for 2 h. Upon completion, the reaction mixture was filtered through celite, evaporated and purified through silica gel column chromatography (ethyl acetate/hexane=1/5, v/v) to obtain compound 46 in 85% yield. ¹H NMR (400 MHz, Chloroform-d) δ 8.12 (ddd, J=5.7, 3.0, 1.6 Hz, 2H), 8.03 (s, 1H), 8.01 (d, J=1.3 Hz, 1H), 7.82-7.79 (m, 1H), 7.77-7.74 (m, 1H), 7.67-7.56 (m, 15H), 7.48-7.43 (m, 7H), 7.40-7.37 (m, 7H), 7.35-7.33 (m, 4H), 7.31-7.28 (m, 14H), 7.25-7.21 (m, 5H), 7.17 (d, J=7.5 Hz, 2H), 7.14-7.10 (m, 4H), 5.62 (s, 1H), 5.38 (s, 1H), 5.34 (s, 1H), 5.18 (s, 1H), 4.94-4.83 (m, 3H), 4.79 (d, J=10.8 Hz, 1H), 4.75 (d, J=4.0 Hz, 1H), 4.72 (d, J=4.1 Hz, 1H), 4.70 (d, J=2.4 Hz, 1H), 4.67 (d, J=3.2 Hz, 1H), 4.64 (d, J=3.5 Hz, 1H), 4.56 (d, J=10.6 Hz, 1H), 4.47 (d, J=3.8 Hz, 1H), 4.38 (dd, J=5.5, 3.2 Hz, 1H), 4.36-4.32 (m, 2H), 4.14-4.08 (m, 2H), 4.05-4.02 (m, 3H), 3.99 (t, J=5.4 Hz, 1H), 3.91 (dd, J=5.6, 1.9 Hz, 1H), 3.88-3.85 (m, 2H), 3.83 (t, J=3.4 Hz, 1H), 3.79 (d, J=10.2 Hz, 1H), 3.74 (d, J=9.9 Hz, 1H), 3.71-3.66 (m, 2H), 3.63-3.57 (m, 2H), 3.54-3.51 (m, 1H), 3.47 (s, 1H), 3.28 (d, J=3.5 Hz, 1H), 3.26 (d, J=3.6 Hz, 1H), 1.86 (s, 3H), 1.20 (s, 3H), 1.02 (s, 9H), 0.97 (s, 9H). ¹³C NMR (101 MHz, Chloroform-d) δ 170.26, 169.96, 165.86, 165.70, 138.18, 137.54, 137.31, 135.90, 135.87, 135.65, 135.59, 135.46, 135.45, 135.23, 133.46, 133.39, 133.28, 133.26, 133.21, 133.14, 133.02, 133.00, 132.70, 131.73, 129.90, 129.82, 129.76, 129.71, 129.66, 129.63, 128.91, 128.69, 128.60, 128.47, 128.43, 128.36, 128.25, 128.18, 128.12, 128.09, 127.98, 127.85, 127.82, 127.76, 127.75, 127.70, 127.66, 127.63, 127.52, 127.40, 127.34, 126.93, 126.10, 126.03, 125.99, 125.94, 125.85, 125.69, 125.57, 98.16, 98.03, 96.96, 85.84, 80.36, 79.15, 77.63, 77.25, 75.30, 75.04, 74.97, 74.22, 73.28, 72.91, 72.83, 72.80, 72.59, 72.42, 71.58, 69.80, 68.54, 66.30, 64.62, 64.46, 64.06, 62.89, 62.27, 61.93, 26.87, 26.80, 20.65, 19.95, 19.44, 19.33. HR-ESI-MS (m/z): [M+Na]⁺ calcd for C₁₂₃H₁₂₆N₆O₂₂SSi₂Na, 2149.8080; found, 2149.8088.

### N-benzyloxycarbonyl-3-aminopropyl-O-[(2-azido-4-O-benzyl-6-O-tert-butyldiphenylsilyl-3-O-(2-naphthylmethyl)-2-deoxy-α-D-glucopyranosyl)-(1→4)-O-(2-O-benzoyl-3-O-benzyl-6-O-acetyl-α-L-idopyranosyl)-(1→4)-O-(2-azido-6-O-tert-butyldiphenylsilyl-3-O-(2-naphthylmethyl)-2-deoxy-α-D-glucopyranosyl)]-(1→4)-O-2-O-benzoyl-3-O-benzyl-6-O-acetyl-α-L-idopyranoside (47)

A solution of donor 46 (0.90 g, 0.42 mmol) and linker benzyl (3-hydroxypropyl) carbamate (0.097 g, 0.46 mmol) in CH₂Cl₂ (15 mL) was stirred under N₂ atmosphere in round bottom flask containing freshly dried 4 Å molecular sieves for 2 h. Next, NIS (0.15 g, 0.67 mmol) and TfOH (7.4 μL, 0.084 mmol) was added at room temperature and reaction completion was monitored by TLC, quenched using few drops of Et3N. Molecular sieves were filtered using celite and organic layer was washed with aqueous Na₂S₂O₃ and brine. The collected organic layer was dried over Na₂SO₄, filtered, concentrated and purified through silica gel column chromatography (ethyl acetate/hexane=1/3, v/v) to obtain compound 47 in 90% yield. ¹H NMR (400 MHz, Chloroform-d) δ 8.09 (dd, J=6.4, 3.1 Hz, 2H), 8.01-7.99 (m, 1H), 7.82-7.78 (m, 1H), 7.76-7.74 (m, 1H), 7.67-7.54 (m, 14H), 7.49 (s, 1H), 7.47-7.45 (m, 2H), 7.43 (s, 1H), 7.40 (s, 1H), 7.39 (s, 2H), 7.37 (s, 2H), 7.34-7.27 (m, 24H), 7.24-7.20 (m, 6H), 7.17-7.11 (m, 6H), 5.63 (s, 1H), 5.37 (s, 1H), 5.18 (s, 1H), 5.08 (s, 2H), 5.03 (s, 1H), 4.96 (s, 1H), 4.88 (dd, J=11.4, 4.5 Hz, 1H), 4.78 (d, J=10.7 Hz, 2H), 4.74 (d, J=11.3 Hz, 2H), 4.68 (d, J=10.8 Hz, 1H), 4.64 (d, J=3.6 Hz, 1H), 4.58 (dd, J=17.4, 11.3 Hz, 2H), 4.39 (dt, J=8.6, 4.4 Hz, 1H), 4.33 (dt, J=11.2, 3.9 Hz, 2H), 4.27-4.24 (m, 2H), 4.10 (t, J=9.4 Hz, 2H), 4.03 (d, J=12.5 Hz, 1H), 3.99-3.93 (m, 3H), 3.89 (s, 1H), 3.87-3.80 (m, 5H), 3.74 (d, J=11.9 Hz, 1H), 3.68 (t, J=9.7 Hz, 2H), 3.61 (d, J=10.3 Hz, 2H), 3.57-3.54 (m, 3H), 3.47 (s, 1H), 3.39 (s, 1H), 3.28 (d, J=3.6 Hz, 1H), 3.25 (d, J=3.3 Hz, 1H), 3.24-3.18 (m, 2H), 1.86 (s, 3H), 1.18 (s, 3H), 1.01 (s, 9H), 0.97 (s, 9H). ¹³C NMR (101 MHz, Chloroform-d) δ 170.37, 169.91, 165.79, 165.68, 156.55, 138.18, 137.54, 137.50, 136.72, 135.88, 135.86, 135.61, 135.58, 135.47, 135.23, 133.46, 133.42, 133.28, 133.23, 133.19, 133.14, 133.01, 132.70, 129.88, 129.84, 129.74, 129.69, 129.64, 129.63, 129.61, 129.59, 128.70, 128.66, 128.58, 128.54, 128.45, 128.38, 128.34, 128.30, 128.25, 128.17, 128.13, 128.10, 128.01, 127.97, 127.82, 127.74, 127.72, 127.68, 127.64, 127.61, 127.58, 127.51, 127.40, 127.34, 126.91, 126.08, 126.01, 125.95, 125.92, 125.84, 125.65, 125.56, 98.15, 98.11, 97.84, 96.93, 80.36, 79.09, 77.63, 77.24, 75.28, 75.02, 74.97, 73.82, 73.25, 73.16, 72.89, 72.80, 72.56, 72.50, 72.40, 72.38, 71.63, 68.79, 68.56, 67.01, 66.57, 65.12, 64.59, 64.42, 64.07, 63.04, 62.34, 62.21, 61.94, 39.64, 29.41, 26.86, 26.78, 20.63, 19.90, 19.44, 19.32. HR-ESI-MS (m/z): [M+Na]⁺ calcd for C₁₂₈H₁₃₅N₇O₂₅Si₂Na, 2248.8944; found, 2248.9016.

### N-benzyloxycarbonyl-3-aminopropyl-O-[(2-azido-4-O-benzyl-6-O-tert-butyldiphenylsilyl-3-O-(2-naphthylmethyl)-2-deoxy-α-D-glucopyranosyl)-(1→4)-O-(3-O-benzyl-α-L-idopyranosyl)-(1→4)-O-(2-azido-6-O-tert-butyldiphenylsilyl-3-O-(2-naphthylmethyl)-2-deoxy-α-D-glucopyranosyl)]-(1→4)-O-3-O-benzyl-α-L-idopyranoside (48)

To a solution of compound 47 (0.84 g, 0.37 mmol) in CH₂Cl₂ (7 mL) and MeOH (7 mL) was added NaOMe (0.06 g, 1.11 mmol) and stirred at room temperature. After 12 h, reaction mixture was quenched using Amberlite IR 120H⁺ resin, filtered, evaporated and purified through silica gel column chromatography (ethyl acetate/hexane=1/2.5, v/v) to obtain compound 48 in 95% yield. ¹H NMR (400 MHz, Chloroform-d) δ 7.81-7.68 (m, 13H), 7.57 (d, J=8.5 Hz, 2H), 7.51-7.44 (m, 15H), 7.41-7.35 (m, 11H), 7.33-7.30 (m, 4H), 7.29-7.27 (m, 6H), 7.17-7.14 (m, 3H), 5.72 (s, 1H), 5.10 (s, 1H), 5.05 (s, 3H), 5.01-4.94 (m, 2H), 4.88-4.84 (m, 2H), 4.80 (dd, J=10.6, 4.7 Hz, 2H), 4.73 (d, J=11.3 Hz, 2H), 4.68 (dd, J=10.9, 3.7 Hz, 2H), 4.63-4.51 (m, 2H), 4.25 (dt, J=14.0, 6.9 Hz, 2H), 3.99 (t, J=9.5 Hz, 1H), 3.89-3.83 (m, 7H), 3.81-3.71 (m, 8H), 3.61-3.54 (m, 8H), 3.48 (d, J=11.5 Hz, 1H), 3.36 (d, J=8.3 Hz, 1H), 3.22 (ddt, J=18.0, 13.6, 5.6 Hz, 2H), 2.70 (dt, J=10.7, 5.2 Hz, 1H), 2.16 (s, 1H), 1.85 (s, 2H), 1.10 (s, 9H), 1.05 (s, 9H). ¹³C NMR (101 MHz, Chloroform-d) δ 156.68, 138.02, 137.50, 137.48, 136.61, 136.00, 135.84, 135.79, 135.60, 135.10, 134.86, 133.44, 133.32, 133.30, 133.15, 133.14, 133.08, 132.84, 132.64, 129.87, 129.82, 129.79, 129.69, 128.72, 128.51, 128.48, 128.45, 128.43, 128.27, 128.07, 128.04, 128.02, 127.90, 127.84, 127.79, 127.70, 127.67, 127.60, 127.56, 127.52, 127.08, 126.61, 126.32, 126.07, 126.00, 125.59, 100.99, 100.07, 94.79, 94.36, 81.25, 79.86, 77.75, 77.26, 76.02, 75.52, 75.06, 73.64, 72.94, 72.47, 72.32, 72.02, 71.69, 70.87, 70.30, 69.56, 66.99, 66.95, 66.88, 66.86, 66.85, 66.63, 66.51, 66.36, 64.61, 63.99, 62.76, 62.49, 61.79, 61.44, 39.68, 29.72, 26.93, 26.83, 19.46, 19.31. HR-ESI-MS (m/z): [M+Na]⁺ calcd for C₁₁₀H₁₂₃N₇O₂₁Si₂Na, 1957.8242; found, 1957.8326.

### N-benzyloxycarbonyl-3-aminopropyl-O-[(2-azido-4-O-benzyl-6-O-tert-butyldiphenylsilyl-3-O-(2-naphthylmethyl)-2-deoxy-α-D-glucopyranosyl)-(1→4)-O-(3-O-benzyl-α-L-idopyranosylurono-6,2-lactone)-(1→4)-O-(2-azido-6-O-tert-butyldiphenylsilyl-3-O-(2-naphthylmethyl)-2-deoxy-α-D-glucopyranosyl)]-(1→4)-O-3-O-benzyl-α-L-idopyranosidurono-6,2-lactone (49)

To a solution of compound 48 (0.67 g, 0.34 mmol) in CH₂Cl₂ (5 mL) and H₂O (5 mL) was added TEMPO (0.01 g, 0.07 mmol), BAIB (0.54 g, 1.70 mmol) and stirred at room temperature. After 16 h, the reaction mixture was extracted using saturated aqueous NH₄Cl. The collected organic layer was dried over Na₂SO₄, filtered, concentrated and purified through silica gel column chromatography (ethyl acetate/hexane=1/3, v/v) to obtain compound 49 in 65% yield. ¹H NMR (400 MHz, Chloroform-d) δ 7.89-7.78 (m, 7H), 7.71-7.68 (m, 4H), 7.65-7.61 (m, 4H), 7.51-7.29 (m, 36H), 7.22-7.17 (m, 3H), 5.84 (s, 1H), 5.47 (s, 1H), 5.25 (d, J=12.0 Hz, 1H), 5.20-5.05 (m, 4H), 4.99 (d, J=10.7 Hz, 1H), 4.95 (s, 1H), 4.92 (s, 1H), 4.87 (d, J=11.6 Hz, 1H), 4.82 (d, J=11.5 Hz, 1H), 4.76 (d, J=8.9 Hz, 1H), 4.70 (d, J=20.3 Hz, 1H), 4.65-4.62 (m, 1H), 4.49 (d, J=12.4 Hz, 2H), 4.32 (d, J=2.8 Hz, 1H), 4.24 (s, 1H), 4.21-4.14 (m, 3H), 4.01-3.85 (m, 5H), 3.82-3.76 (m, 3H), 3.71 (dt, J=8.0, 3.3 Hz, 3H), 3.65-3.59 (m, 3H), 3.53 (dd, J=14.1, 3.7 Hz, 1H), 3.48-3.45 (m, 2H), 3.34 (dt, J=13.6, 6.6 Hz, 1H), 2.77 (dd, J=9.4, 4.4 Hz, 1H), 1.89-1.87 (m, 2H), 1.07 (s, 18H). ¹³C NMR (101 MHz, Chloroform-d) δ 167.43, 167.04, 156.60, 138.13, 137.02, 136.86, 136.70, 136.21, 136.00, 135.88, 135.58, 135.56, 135.19, 133.65, 133.34, 133.30, 133.14, 133.01, 132.77, 132.74, 130.02, 129.85, 129.73, 129.70, 129.64, 128.52, 128.49, 128.42, 128.32, 128.23, 128.21, 128.19, 128.11, 128.08, 128.03, 127.93, 127.81, 127.70, 127.65, 127.53, 127.52, 127.19, 126.49, 126.19, 126.09, 126.02, 125.91, 124.87, 124.68, 99.84, 98.98, 96.78, 96.74, 80.99, 80.27, 80.00, 78.32, 78.06, 77.87, 77.77, 77.24, 76.10, 75.82, 74.95, 74.34, 72.65, 72.57, 72.49, 72.35, 72.25, 71.82, 69.42, 69.37, 69.11, 66.64, 63.34, 61.69, 61.47, 39.89, 29.48, 26.91, 19.36, 19.28. HR-ESI-MS (m/z): [M+Na]⁺ calcd for C₁₁₀H₁₁₅N₇O₂₁Si₂Na, 1949.7616; found, 1949.7651.

### N-benzyloxycarbonyl-3-aminopropyl-O-[(2-acetamido-4-O-benzyl-6-O-tert-butyldiphenylsilyl-3-O-(2-naphthylmethyl)-2-deoxy-α-D-glucopyranosyl)-(1→4)-O-(3-O-benzyl-α-L-idopyranosylurono-6,2-lactone)-(1→4)-O-(2-acetamido-6-O-tert-butyldiphenylsilyl-3-O-(2-naphthylmethyl)-2-deoxy-α-D-glucopyranosyl)]-(1→4)-O-3-O-benzyl-α-L-idopyranosidurono-6,2-lactone (50)

To a solution of compound 49 (0.40 g, 0.20 mmol) in THF (3 mL), AcOH (2 mL) and Ac₂O (2 mL) was added Zn dust (0.52 g, 8 mmol) and stirred for 12 h at room temperature. Upon completion, the reaction mixture was filtered through celite and volatiles were evaporated. The remaining residue was extracted with ethyl acetate, saturated NaHCO₃ and washed with brine. The collected organic layer was dried over Na₂SO₄, filtered, concentrated and purified through silica gel column chromatography (ethyl acetate/hexane=1/2.5, v/v) to obtain compound 50 in 70% yield. ¹H NMR (400 MHz, Chloroform-d) δ 7.92-7.86 (m, 3H), 7.82-7.81 (m, 2H), 7.78 (d, J=8.5 Hz, 1H), 7.76-7.74 (m, 1H), 7.71-7.70 (m, 2H), 7.69-7.68 (m, 3H), 7.68-7.66 (m, 2H), 7.65 (d, J=1.3 Hz, 1H), 7.58 (s, 1H), 7.53 (dt, J=6.1, 2.7 Hz, 2H), 7.51-7.49 (m, 1H), 7.48-7.46 (m, 2H), 7.45-7.42 (m, 3H), 7.41 (s, 2H), 7.39-7.38 (m, 2H), 7.38-7.37 (m, 1H), 7.35-7.31 (m, 7H), 7.29 (s, 1H), 7.28-7.27 (m, 7H), 7.26-7.24 (m, 3H), 7.23-7.20 (m, 3H), 7.18-7.16 (m, 2H), 7.05 (d, J=7.3 Hz, 2H), 5.50 (s, 1H), 5.42 (t, J=5.2 Hz, 1H), 5.16 (d, J=3.6 Hz, 1H), 5.07-5.03 (m, 4H), 5.00 (s, 1H), 4.93-4.90 (m, 3H), 4.80 (d, J=3.5 Hz, 1H), 4.77 (d, J=2.1 Hz, 1H), 4.73 (d, J=6.9 Hz, 1H), 4.63 (d, J=12.4 Hz, 1H), 4.48 (s, 1H), 4.42 (d, J=3.4 Hz, 1H), 4.37 (s, 1H), 4.33 (d, J=2.5 Hz, 2H), 4.30 (d, J=5.4 Hz, 1H), 4.27-4.21 (m, 1H), 4.09 (qd, J=6.2, 4.2, 3.7 Hz, 3H), 4.02-3.89 (m, 7H), 3.86-3.70 (m, 2H), 3.68-3.59 (m, 5H), 3.53 (q, J=10.8, 9.0 Hz, 2H), 3.36 (dt, J=11.3, 6.0 Hz, 1H), 3.27 (dt, J=13.8, 6.7 Hz, 1H), 1.83-1.80 (m, 2H), 1.59 (s, 3H), 1.39 (s, 3H), 1.09 (s, 9H), 1.06 (s, 9H). ¹³C NMR (101 MHz, Chloroform-d) δ 169.88, 169.77, 167.57, 167.18, 156.46, 138.08, 136.62, 136.59, 135.92, 135.89, 135.78, 135.64, 135.57, 133.55, 133.28, 133.20, 133.08, 133.02, 132.94, 132.85, 132.54, 129.96, 129.83, 129.63, 128.64, 128.50, 128.46, 128.33, 128.29, 128.22, 128.09, 128.04, 127.93, 127.90, 127.78, 127.71, 127.66, 127.52, 127.16, 126.58, 126.44, 126.38, 126.34, 126.28, 126.26, 98.94, 98.73, 98.38, 96.61, 79.70, 79.39, 79.08, 79.00, 78.65, 78.28, 78.04, 77.29, 76.88, 75.09, 74.98, 74.79, 72.84, 72.28, 72.22, 72.13, 71.74, 69.35, 68.57, 66.60, 61.94, 61.70, 52.92, 52.80, 39.16, 29.61, 26.94, 26.87, 23.17, 22.87, 19.26, 19.20. HR-ESI-MS (m/z): [M+H]⁺ calcd for C₁₁₄H₁₂₄N₃O₂₃Si₂, 1958.8164; found, 1958.8163.

### General Procedure for Benzyl Ester Formation:

To a solution of starting material (1 mmol) in THF/H₂O (1/1) was added LiOH. H₂O (3 mmol) and stirred at room temperature for 2 h. Upon completion, the reaction mixture was quenched using Amberlite IR 120H⁺ resin, filtered, evaporated and dried. Next, the residue was dissolved in DMF and added BnBr (4 mmol), TBAI (1.4 mmol), NaHCO₃ (5 mmol) and stirred at 60° C. under N₂ atmosphere. After 2 h, the reaction mixture was extracted with ethyl acetate and washed with brine. The organic layer obtained was dried over Na₂SO₄, filtered, concentrated and purified through silica gel column chromatography.

### General Procedure for Selective NAP Deprotection

To a solution of starting material (1 mmol) in CH₂Cl₂ (6 mL) and H₂O (340 μL) was added DDQ (5 mmol) portion wise over the interval of 20 min. After 1 h, the reaction mixture was quenched using NaHCO₃, extracted with CH₂Cl₂ and washed with brine. The organic layer obtained was dried over Na₂SO₄, filtered, concentrated and purified through silica gel column chromatography.

### General Procedure for Desilylation Pre O-Sulfation

A solution of starting material (1 mmol) in pyridine (1 mL) was stirred at ice cold temperature under N₂ atmosphere. After 15 minutes, 70% HF.py complex (5 mmol) was added drop wise and reaction mixture was stirred for 12 h. Upon completion, the mixture was extracted with ethyl acetate and washed with 1 N HCl and brine. The combined organic layer was dried over Na₂SO₄, filtered, concentrated and purified through silica gel column chromatography.

### General Procedure for O-Sulfation

To a solution of starting material (1 mmol) in DMF (2 mL) was added SO₃·NEt₃ complex (10 mmol per-OH) under N₂ atmosphere and stirred at 60° C. for 3 days. Upon completion of reaction, aqueous NaHCO₃ (20 mmol) was added to reaction mixture and stirred for another 16 h. Next, the reaction mixture was concentrated under reduced pressure and the resulting residue was filtered with MeOH through whatman filter paper. The filtrate was concentrated and purified using silica column chromatography.

### General Procedure for O-Phophorylation

To a solution of starting material (1 mmol) in CH₂Cl₂ (1 mL) and pyridine (1 mL) was added NEt₃ (10 mmol), DMAP (0.3 mmol) and diphenyl phosphoryl chloride (5-10 mmol) at 0° C. After 12 h, the reaction mixture was diluted with CH₂Cl₂ and extracted with 1N HCl and brine. The combined organic layer was dried over Na₂SO₄, filtered, concentrated and purified through silica gel column chromatography.

### General Procedure for Desilylation Post O-Sulfation

To a solution of starting material (1 mmol) in pyridine (1 mL) was added HF.py (5 mmol) at ice cold temperature. After stirring for 12 h, the volatiles were evaporated and the residue was purified using Sephadex LH 20 column using MeOH as an eluent.

### Global Deprotection

To a solution of starting material (1 mmol) in THF (1 mL) and H₂O (1 mL) was added LiOH·H₂O (10 mmol) and stirred at room temperature for 2 h. Upon completion, the reaction mixture was diluted with MeOH and quenched using Dowex 50WX8 H⁺ resin. The mixture was filtered, concentrated and eluted through Bond Elute C-18 column (H₂O/ACN=1/5, v/v). The combined fraction was concentrated under reduced pressure and dissolved in MeOH for hydrogenolysis. The reaction mixture along with Pd(OH)₂ was stirred under H₂ atm. After 36 h, the mixture was filtered, concentrated and eluted through Bond Elute C-18 column using H₂O as eluent. The combined H₂O fractions were pooled and lyophilized to yield fully deprotected O-sulfated tetra saccharides. Also, for few compounds, after Pd(OH)₂ hydrogenolysis the as obtained product was again kept for hydrogenolysis using PtO₂ as a catalyst under H₂ atm for 24 h. Finally, the reaction mixture was filtered, concentrated under reduced pressure and eluted through Bond Elute C-18 column using H₂O as eluent. The combined H₂O fraction were pooled and lyophilized to yield fully deprotected 6-O-phosphorylated tetrasaccharides.

Example 13: Synthesis of 3-Aminopropyl-O-[(2-amino-2-deoxy-α-D-glucopyranosyl)-(1→4)-O-(2-O-sulfonato-α-L-idopyranosyluronate)-(1→4)-O-(2-amino-2-deoxy-α-D-glucopyranosyl)]-(1→4)-O-2-O-sulfonato-α-L-idopyranosiduronate

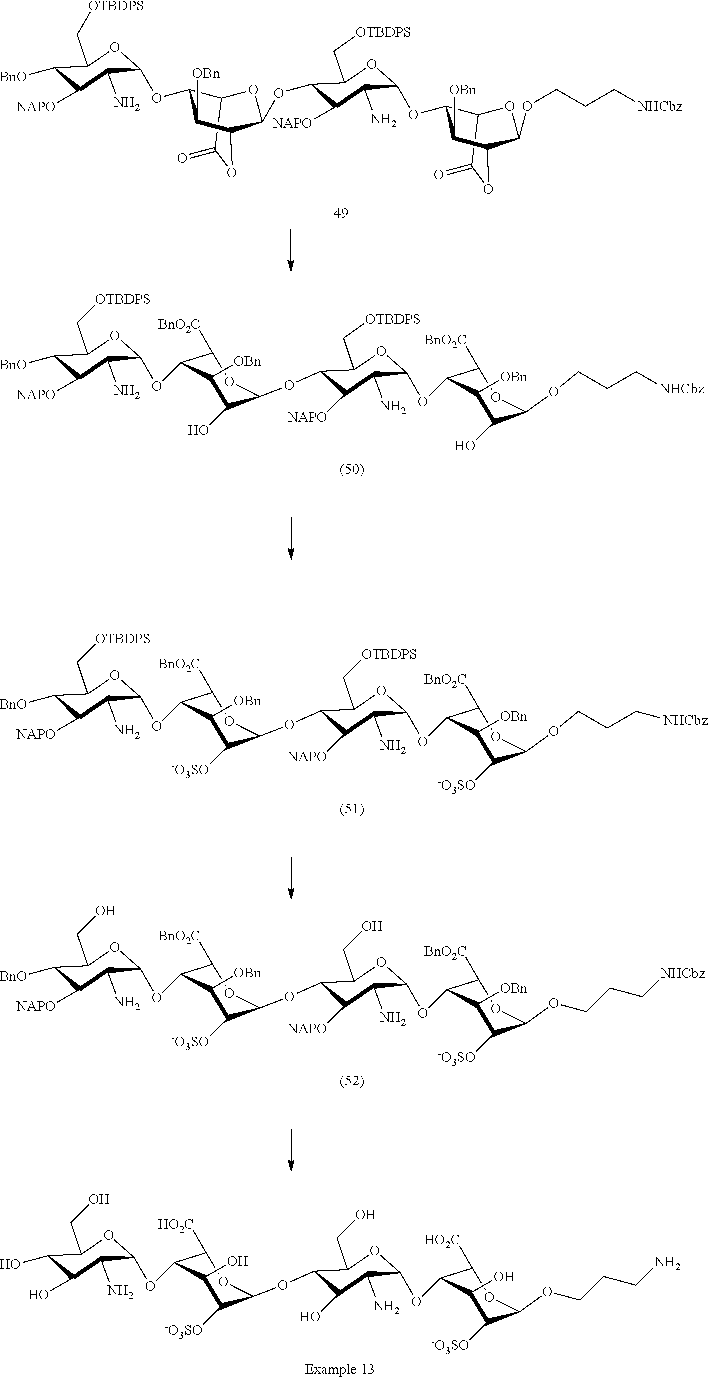

Step-1: N-benzyloxycarbonyl-3-aminopropyl-O-[(benzyl(2-azido-4-O-benzyl-6-O-tert-butyldiphenylsilyl-3-O-(2-naphthylmethyl)-2-deoxy-α-D-glucopyranosyl))-(1→4)-O-(3-O-benzyl-α-L-idopyranosyluronate)-(1→4)-O-(benzyl(2-azido-6-O-tert-butyldiphenylsilyl-3-O-(2-naphthylmethyl)-2-deoxy-α-D-glucopyranosyl))]-(1→4)-O-3-O-benzyl-α-L-idopyranosiduronate (50)

Followed general procedure for benzyl ester formation of compound 49 yielded compound 50 (75%). ¹H NMR (400 MHz, Chloroform-d) δ 7.74-7.66 (m, 3H), 7.65-7.41 (m, 13H), 7.38-7.35 (m, 2H), 7.33-7.11 (m, 37H), 7.07 (ddd, J=13.9, 6.6, 2.9 Hz, 4H), 6.94 (ddt, J=8.6, 3.3, 1.7 Hz, 1H), 6.85 (t, J=7.5 Hz, 2H), 6.77 (d, J=7.2 Hz, 2H), 5.44-5.36 (m, 2H), 5.04 (d, J=12.4 Hz, 1H), 4.96 (d, J=3.6 Hz, 1H), 4.93-4.88 (m, 4H), 4.77-4.73 (m, 4H), 4.70-4.67 (m, 2H), 4.61 (dd, J=12.2, 6.2 Hz, 2H), 4.56 (d, J=2.7 Hz, 2H), 4.53 (d, J=4.4 Hz, 1H), 4.51-4.39 (m, 3H), 4.08 (t, J=9.5 Hz, 1H), 3.95-3.93 (m, 2H), 3.85 (t, J=3.9 Hz, 1H), 3.76 (ddd, J=22.1, 12.9, 7.5 Hz, 5H), 3.68 (d, J=4.5 Hz, 1H), 3.64-3.57 (m, 4H), 3.53-3.47 (m, 3H), 3.43-3.30 (m, 5H), 3.19 (d, J=10.6 Hz, 1H), 3.16-3.11 (m, 1H), 1.78-1.65 (m, 2H), 0.98 (s, 9H), 0.94 (s, 9H). ¹³C NMR (101 MHz, Chloroform-d) δ 169.02, 168.76, 156.46, 138.24, 137.35, 137.32, 136.57, 135.99, 135.88, 135.86, 135.60, 135.19, 135.18, 134.81, 133.47, 133.34, 133.30, 133.26, 133.15, 133.05, 132.75, 129.74, 129.69, 129.67, 128.68, 128.62, 128.51, 128.41, 128.33, 128.27, 128.24, 128.21, 128.12, 128.01, 127.94, 127.92, 127.81, 127.77, 127.72, 127.65, 127.62, 127.55, 127.53, 127.50, 126.95, 126.30, 126.25, 126.05, 126.02, 125.96, 125.71, 125.51, 101.48, 100.99, 95.43, 80.83, 79.48, 79.46, 77.59, 77.24, 75.79, 75.17, 74.93, 74.29, 73.33, 72.96, 72.55, 72.22, 71.56, 71.39, 71.24, 68.44, 68.22, 68.00, 67.99, 67.21, 67.09, 67.08, 67.00, 66.64, 66.61, 64.15, 63.81, 62.13, 61.68, 39.83, 29.45, 26.94, 26.88. HR-ESI-MS (m/z): [M+Na]⁺ calcd for C₁₂₄H₁₃₁N₇O₂₃Si₂Na, 2165.8766; found, 2165.8772.

Step-2: N-benzyloxycarbonyl-3-aminopropyl-O-[(benzyl(2-azido-4-O-benzyl-6-0-tert-butyldiphenylsilyl-3-O-(2-naphthylmethyl)-2-deoxy-α-D-glucopyranosyl))-(1→4)-O-(3-O-benzyl-2-O-sulfonato-α-L-idopyranosyluronate)-(1→4)-O-(benzyl(2-azido-6-O-tert-butyldiphenylsilyl-3-O-(2-naphthylmethyl)-2-deoxy-α-D-glucopyranosyl))]-(1→4)-O-3-O-benzyl-2-O-sulfonato-α-L-idopyranosiduronate (51)

Followed general procedure for O-sulfation of compound 50 yielded compound 51 (68%). ¹H NMR (400 MHz, Methanol-d4) δ 7.79-7.76 (m, 7H), 7.74-7.70 (m, 3H), 7.61-7.41 (m, 13H), 7.38-7.30 (m, 14H), 7.29-7.15 (m, 23H), 7.04 (t, J=6.6 Hz, 2H), 6.96 (t, J=7.5 Hz, 2H), 6.65 (d, J=7.7 Hz, 1H), 5.71 (s, 1H), 5.37 (d, J=12.1 Hz, 1H), 5.22 (d, J=3.5 Hz, 1H), 5.17 (s, 2H), 5.06 (s, 1H), 5.02-4.89 (m, 7H), 4.83 (d, J=5.6 Hz, 1H), 4.77-4.69 (m, 4H), 4.63-4.51 (m, 3H), 4.48 (s, 1H), 4.38 (d, J=11.7 Hz, 1H), 4.33-4.26 (m, 2H), 4.22-4.19 (m, 2H), 4.04-3.98 (m, 3H), 3.86 (d, J=10.4 Hz, 2H), 3.82-3.70 (m, 4H), 3.65-3.54 (m, 4H), 3.29-3.12 (m, 3H), 1.81-1.73 (m, 2H), 0.99 (d, J=6.8 Hz, 18H). ¹³C NMR (101 MHz, Methanol-d₄) δ 169.75, 169.00, 157.34, 138.43, 137.89, 137.83, 136.89, 135.91, 135.79, 135.69, 135.41, 134.97, 133.67, 133.35, 133.25, 133.03, 132.78, 129.45, 129.20, 128.45, 128.15, 128.09, 128.04, 127.83, 127.47, 127.38, 127.22, 126.97, 126.45, 125.95, 125.56, 125.41, 125.36, 125.22, 125.07, 99.44, 98.67, 96.01, 94.54, 79.41, 78.57, 77.75, 74.81, 74.28, 74.15, 73.06, 72.48, 72.31, 72.03, 71.88, 71.38, 70.72, 69.27, 67.89, 67.22, 66.95, 66.35, 65.94, 63.89, 63.75, 62.39, 62.06, 37.92, 29.35, 29.16, 26.31, 26.05. HR-ESI-MS (m/z): [M−2H]²⁻ calcd for C₁₂₄H₁₂₉N₇O₂₉S₂Si₂²⁻, 1150.3930; found, 1150.3950.

Step-3: N-benzyloxycarbonyl-3-aminopropyl-O-[(benzyl(2-azido-4-O-benzyl-3-O-(2-naphthylmethyl)-2-deoxy-α-D-glucopyranosyl))-(1→4)-O-(3-O-benzyl-2-O-sulfonato-α-L-idopyranosyluronate)-(1→4)-O-(benzyl(2-azido-3-O-(2-naphthylmethyl)-2-deoxy-α-D-glucopyranosyl))]-(1→4)-O-3-O-benzyl-2-O-sulfonato-α-L-idopyranosiduronate (52)

Followed general procedure for desilylation of compound 51 yielded compound 52 (68%). ¹H NMR (400 MHz, Methanol-d4) δ 7.82-7.78 (m, 1H), 7.76-7.74 (m, 2H), 7.72-7.70 (m, 2H), 7.63 (dd, J=16.5, 8.2 Hz, 2H), 7.47-7.39 (m, 10H), 7.36-7.34 (m, 5H), 7.32-7.27 (m, 10H), 7.26-7.24 (m, 5H), 7.17-7.10 (m, 3H), 7.02 (t, J=7.5 Hz, 2H), 6.62 (d, J=7.5 Hz, 2H), 5.51 (s, 1H), 5.41 (d, J=12.1 Hz, 1H), 5.27-5.26 (m, 1H), 5.20 (s, 1H), 5.15 (d, J=3.0 Hz, 1H), 5.09-5.06 (m, 2H), 5.02-4.97 (m, 4H), 4.94-4.89 (m, 2H), 4.79-4.69 (m, 3H), 4.64-4.62 (m, 1H), 4.62-4.50 (m, 4H), 4.39-4.35 (m, 3H), 4.18-4.14 (m, 1H), 4.12-4.03 (m, 2H), 3.99-3.73 (m, 7H), 3.61-3.51 (m, 3H), 3.48-3.38 (m, 3H), 3.31-3.13 (m, 4H), 1.84-1.76 (m, 2H). ¹³C NMR (101 MHz, Methanol-d₄) δ 174.15, 169.81, 157.39, 138.37, 137.87, 137.71, 136.94, 135.77, 135.69, 135.07, 134.51, 133.33, 133.31, 133.04, 132.85, 128.51, 128.41, 128.27, 128.25, 128.20, 128.13, 128.06, 127.95, 127.89, 127.82, 127.75, 127.68, 127.60, 127.58, 127.55, 127.54, 127.39, 127.37, 127.31, 127.24, 127.18, 126.39, 125.86, 125.62, 125.49, 125.45, 125.22, 125.09, 99.55, 98.87, 95.46, 95.27, 79.55, 78.42, 77.42, 74.73, 74.36, 74.06, 73.69, 72.81, 72.32, 72.19, 72.08, 71.28, 71.25, 70.76, 70.26, 69.98, 69.74, 68.03, 67.49, 67.26, 66.50, 66.39, 65.96, 65.07, 63.71, 62.66, 59.83, 59.72, 37.99, 33.56, 31.67, 29.37, 29.35, 29.31, 29.20, 29.07, 29.01, 28.81, 24.60, 22.34, 13.07, 7.89. HR-ESI-MS (m/z): [M−2H]²⁻ calcd for C₉₂H₉₃N₇O₂₉S₂²⁻, 911.7735; found, 911.7748.

Step-4:3-aminopropyl-O-[(2-amino-2-deoxy-α-D-glucopyranosyl)-(1→4)-O-(2-O-sulfonato-α-L-idopyranosyluronate)-(1→4)-O-(2-amino-2-deoxy-α-D-glucopyranosyl)]-(1→4)-O-2-O-sulfonato-α-L-idopyranosiduronate

Followed general procedure for global deprotection of compound 52 yielded Example 13 (72%). ¹H NMR (600 MHz, Deuterium Oxide) δ 5.33 (s, 1H), 5.15 (s, 1H), 5.05 (s, 1H), 4.84 (s, 1H), 4.46 (s, 1H), 4.28-4.25 (m, 3H), 4.17 (s, 1H), 4.06 (d, J=14.2 Hz, 2H), 3.88 (t, J=9.9 Hz, 1H), 3.81-3.77 (m, 5H), 3.73 (s, 2H), 3.68-3.67 (m, 2H), 3.64-3.62 (m, 1H), 3.40 (t, J=9.6 Hz, 1H), 3.29 (d, J=10.7 Hz, 1H), 3.26-3.24 (m, 1H), 3.09-3.05 (m, 2H), 1.96-1.87 (m, 2H). ¹³C NMR (151 MHz, Deuterium Oxide) δ 175.38, 174.85, 98.74, 97.78, 91.06, 90.96, 76.07, 72.53, 72.42, 72.01, 71.44, 70.06, 69.95, 69.33, 69.06, 67.99, 66.87, 66.59, 66.43, 62.40, 62.37, 59.71, 59.27, 54.50, 54.14, 38.32, 26.06. HR-ESI-MS (m/z): [M−2H]²⁻ calcd for C₂₇H₄₅N₃O₂₇S₂²⁻, 453.5846; found, 453.5911.

Examples 14-35: The following examples 14-35 were synthesised by following the above experimental procedures with appropriate starting materials and non-critical variations.

| Ex. | Structure & Chemical name | NMR & Mass |
|---|---|---|
| 14 | 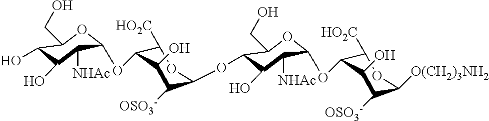 3-aminopropyl-O-[(2-acetamido-2-deoxy-α-D-glucopyranosyl)-(1→4)-O-(2-O-sulfonato- α-L-idopyranosyluronate)-(1→4)-O-(2-acetamido-2-deoxy-α-D-glucopyranosyl)]- (1→4)-O-2-O-sulfonato-α-L-idopyranosiduronate | ¹H NMR (600 MHz, Deuterium Oxide) δ 5.12 (s, 2H), 5.06 (s, 1H), 5.03 (d, J = 3.4 Hz, 1H), 5.01 (d, J = 3.4 Hz, 1H), 4.76 (d, J = 1.8 Hz, 1H), 4.21 (d, J = 6.5 Hz, 2H), 4.17 (d, J = 13.4 Hz, 2H), 3.97 (d, J = 11.1 Hz, 2H), 3.90 (ddd, J = 18.5, 10.5, 3.4 Hz, 2H), 3.85- 3.81 (m, 1H), 3.76 (s, 2H), 3.71 (d, J = 2.8 Hz, 2H), 3.65-3.59 (m, 4H), 3.56-3.50 (m, 2H), 3.42 (d, J = 9.2 Hz, 1H), 3.06 (t, J = 6.3 Hz, 2H), 1.98 (s, 3H), 1.96 (s, 3H), 1.93-1.88 (m, |
| | | 2H). ¹³C NMR (151 MHz, Deuterium |
| | | Oxide) δ 174.80, 173.03, 172.79, |
| | | 99.17, 98.34, 94.86, 94.32, 76.95, |
| | | 73.19, 73.15, 72.36, 71.60, 71.36, |
| | | 71.05, 70.94, 69.70, 69.34, 66.72, |
| | | 66.19, 63.97, 63.42, 60.02, 59.47, |
| | | 53.51, 53.22, 38.15, 26.02, 22.14. HR- |
| | | ESI-MS (m/z): [M − 2H]²⁻ calcd for |
| | | C₃₁H₄₉N₃O₂₉S₂²⁻, 495.5952; found, |
| | | 495.5922. |
| 15 | 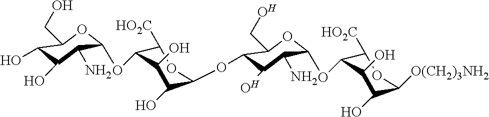 3-aminopropyl-O-[(2-amino-2-deoxy-α-D-glucopyranosyl)-(1→4)-O-(α-L- idopyranosyluronate)-(1→4)-O-(2-amino-2-deoxy-α-D-glucopyranosyl)]-(1→4)-O-α-L- idopyranosiduronate | ¹H NMR (400 MHz, Deuterium Oxide) δ 5.37 (d, J = 3.6 Hz, 1H), 5.29 (d, J = 3.7 Hz, 1H), 4.72 (d, J = 4.2 Hz, 1H), 4.59 (d, J = 3.2 Hz, 1H), 4.36 (d, J = 2.7 Hz, 1H), 4.03-4.01 (m, 1H), 3.96 (t, J = 3.1 Hz, 1H), 3.91-3.88 (m, 1H), 3.79 (dd, J = 7.0, 3.8 Hz, 1H), 3.76-3.72 (m, 2H), 3.70 (s, 3H), 3.68- 3.66 (m, 3H), 3.65-3.63 (m, 1H), 3.61 (d, J = 9.4 Hz, 1H), 3.57-3.56 (m, 1H), 3.55-3.51 (m, 3H), 3.32 (t, J = 9.4 Hz, 1H), 3.19 (ddd, J = 12.6, 10.6, 3.6 Hz, |
| | | 2H), 2.97 (t, J = 6.7 Hz, 2H), 1.87- |
| | | 1.78 (m, 2H). ¹³C NMR (101 MHz, |
| | | Deuterium Oxide) δ 174.73, 174.51, |
| | | 101.85, 100.65, 92.25, 92.22, 75.58, |
| | | 74.41, 73.27, 72.32, 71.49, 70.12, |
| | | 70.10, 69.42, 69.23, 68.69, 68.41, |
| | | 68.12, 67.59, 66.35, 59.88, 59.30, |
| | | 54.23, 53.97, 38.03, 26.36. HR-ESI- |
| | | MS (m/z): [M + H]⁺ calcd for |
| | | C₂₇H₄₇N₃O₂₁⁺, 750.2775; found, |
| | | 750.2780. |
| 16 | 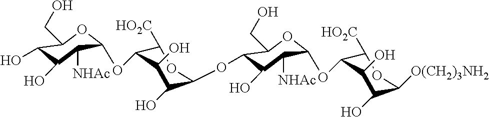 3-aminopropyl-O-[(2-acetamido-2-deoxy-α-D-glucopyranosyl)-(1→4)-O-(α-L- | ¹H NMR (400 MHz, Deuterium Oxide) δ 5.08 (d, J = 3.7 Hz, 1H), 5.05 (d, J = 3.8 Hz, 1H), 5.00 (d, J = 2.7 Hz, 1H), 4.90 (d, J = 2.7 Hz, 1H), 4.84 (d, J = 1.8 Hz, 1H), 4.67 (d, J = 2.4 Hz, 1H), 4.02 (t, J = 3.1 Hz, 1H), 3.99 (t, J = 2.6 Hz, 1H), 3.94 (t, J = 3.6 Hz, 1H), 3.91- 3.79 (m, 4H), 3.75-3.71 (m, 4H), 3.67-3.65 (m, 3H), 3.64-3.58 (m, |
| | idopyranosyluronate)-(1→4)-O-(2-acetamido-2-deoxy-α-D-glucopyranosyl)]- | 3H), 3.58-3.52 (m, 2H), 3.45 (d, J = |
| | (1→4)-O-α-L-idopyranosiduronate | 9.3 Hz, 1H), 3.06 (t, J = 6.6 Hz, 2H), |
| | | 1.95-1.88 (m, 8H). ¹³C NMR (101 |
| | | MHz, Deuterium Oxide) δ 174.43, |
| | | 174.40, 173.41, 173.02, 101.63, |
| | | 100.57, 95.15, 95.00, 76.85, 73.76, |
| | | 73.26, 72.45, 71.49, 71.08, 69.77, |
| | | 69.29, 69.03, 68.07, 68.04, 67.96, |
| | | 67.16, 66.63, 60.03, 59.55, 53.64, |
| | | 53.39, 38.13, 26.24, 21.93. HR-ESI- |
| | | MS (m/z): [M − H] calcd for |
| | | C₃₁H₅₀N₃O₂₃⁻, 832.2840; found, |
| | | 832.2835. |
| 17 | 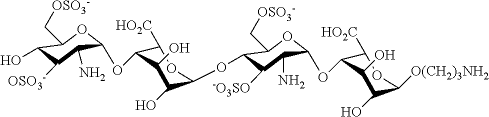 3-aminopropyl-O-[(2-amino-6-O-sulfonato-3-O-sulfonato-2-deoxy-α-D-glucopyranosyl)- (1→4)-O-(α-L-idopyranosyluronate)-(1→4)-0-(2-amino-6-O-sulfonato-3-O-sulfonato-2- deoxy-α-D-glucopyranosyl)]-(1→4)-O-α-L-idopyranosiduronate | 1H NMR (600 MHz, Deuterium Oxide) δ 5.26 (d, J = 3.2 Hz, 1H), 5.09 (d, J = 3.5 Hz, 1H), 4.87 (d, J = 4.2 Hz, 1H), 4.80-4.78 (m, 2H), 4.42 (d, J = 2.7 Hz, 1H), 4.31-4.24 (m, 4H), 4.20-4.17 (m, 1H), 4.13-4.08 (m, 2H), 4.02- 3.99 (m, 3H), 3.96-3.95 (m, 2H), 3.88 (t, J = 9.7 Hz, 1H), 3.84-3.79 (m, 2H), 3.67-3.55 (m, 4H), 3.08-3.04 (m, 4H), 2.99 (dd, J = 10.2, 3.5 Hz, 1H), 1.94-1.87 (m, 2H). ¹³C NMR (151 MHz, Deuterium Oxide) δ 174.98, |
| | | 171.04, 100.49, 100.43, 96.66, 95.49, |
| | | 79.53, 75.67, 74.77, 71.37, 70.91, |
| | | 70.40, 70.14, 70.03, 69.95, 68.98, |
| | | 68.79, 68.40, 67.47, 66.48, 66.29, |
| | | 66.13, 54.46, 53.86, 38.14, 26.39. HR- |
| | | ESI-MS (m/z): [M + H]⁺ calcd for |
| | | C₂₇H₄₈N₃O₃₃S₄⁺, 1070.1048; found, |
| | | 1070.1050. |
| 18 | 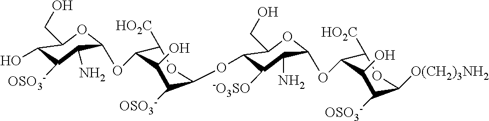 3-aminopropyl-O-[(2-amino-3-O-sulfonato-2-deoxy-α-D-glucopyranosyl)-(1→4)-O-(2- O-sulfonato-α-L-idopyranosyluronate)-(1→4)-O-(2-amino-3-O-sulfonato-2-deoxy-α- D-glucopyranosyl)]-(1→4)-O-2-O-sulfonato-α-L-idopyranosiduronate | ¹H NMR (600 MHz, Deuterium Oxide) δ 5.44 (d, J = 3.6 Hz, 1H), 5.37 (d, J = 3.7 Hz, 1H), 4.83 (d, J = 3.7 Hz, 1H), 4.80 (d, J = 2.4 Hz, 1H), 4.77 (d, J = 3.1 Hz, 1H), 4.52 (d, J = 2.6 Hz, 1H), 4.12-4.11 (m, 1H), 4.06-4.05 (m, 1H), 4.00-3.97 (m, 1H), 3.90 (dd, J = 6.8, 4.1 Hz, 1H), 3.85-3.81 (m, 2H), 3.77-3.72 (m, 6H), 3.70-3.67 (m, 2H), 3.66-3.62 (m, 3H), 3.41 (t, J = 9.6 Hz, 1H), 3.31-3.26 (m, 2H), 3.07- 3.05 (m, 2H), 1.94-1.87 (m, 2H). |
| | | ¹³C NMR (151 MHz, Deuterium |
| | | Oxide) δ 174.23, 173.94, 101.86, |
| | | 100.69, 92.45, 92.34, 75.87, 74.15, |
| | | 73.16, 72.48, 69.81, 69.45, 69.40, |
| | | 69.17, 68.49, 68.09, 68.03, 67.22, |
| | | 66.45, 59.90, 59.32, 54.23, 53.96, |
| | | 38.06, 26.35 HR-ESI-MS (m/z): |
| | | [M + H]⁺ calcd for C₂₇H₄₈N₃O₃₃S₄+, |
| | | 1070.1048; found, 1070.1052. |
| 19 | 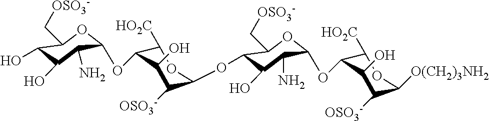 3-aminopropyl-O-[(2-amino-6-O-sulfonato-2-deoxy-α-D-glucopyranosyl)-(1→4)-O-(2- O-sulfonato-α-L-idopyranosyluronate)-(1→4)-O-(2-amino-6-O-sulfonato-2-deoxy-α- D-glucopyranosyl)]-(1→4)-O-2-O-sulfonato-α-L-idopyranosiduronate | ¹H NMR (400 MHz, Deuterium Oxide) δ 5.29 (dd, J = 7.0, 3.7 Hz, 2H), 5.10 (s, 1H), 5.01 (s, 1H), 4.83 (s, 1H), 4.47 (s, 1H), 4.25-4.23 (m, 2H), 4.22 (d, J = 2.0 Hz, 1H), 4.19 (d, J = 3.0 Hz, 1H), 4.17-4.14 (m, 2H), 4.13-4.11 (m, 1H), 4.09 (s, 1H), 4.06 (s, 1H), 4.04 (s, 1H), 3.87 (d, J = 9.6 Hz, 1H), 3.82 (d, J = 10.4 Hz, 1H), 3.80-3.75 (m, 2H), 3.74-3.69 (m, 1H), 3.66 (d, J = 9.7 Hz, 1H), 3.61-3.55 (m, 1H), 3.43 (t, J = 9.7 Hz, 1H), 3.31-3.22 |
| | | (m, 2H), 3.02 (q, J = 6.7, 5.6 Hz, 2H), |
| | | 1.91-1.82 (m, 2H). ¹³C NMR (151 |
| | | MHz, Deuterium Oxide) δ 174.95, |
| | | 98.71, 97.82, 91.30, 90.92, 75.55, |
| | | 72.67, 72.48, 70.32, 70.14, 69.59, |
| | | 69.23, 68.64, 68.10, 67.05, 66.76, |
| | | 66.38, 66.13, 62.72, 62.30, 54.27, |
| | | 53.99, 38.43, 26.08. HR-ESI-MS |
| | | (m/z): [M + H]⁺ calcd for |
| | | C₂₇H₄₈N₃O₃₃S₄⁺, 1070.1048; found, |
| | | 1070.1051. |
| 20 | 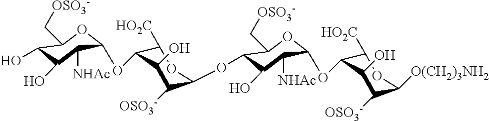 3-aminopropyl-O-[(2-acetamido-6-0-sulfonato-2-deoxy-α-D-glucopyranosyl)- (1→4)-O-(2-O-sulfonato-α-L-idopyranosyluronate)-(1→4)-O-(2- acetamido-6-O-sulfonato-2-deoxy-α-D-glucopyranosyl)]-(1→4)-O-2-O-sulfonato-α- L-idopyranosiduronate | ¹H NMR (400 MHz, Deuterium Oxide) δ 5.14 (s, 1H), 5.10 (d, J = 1.7 Hz, 1H), 5.07 (d, J = 3.7 Hz, 3H), 4.76 (d, J = 2.0 Hz, 1H), 4.28-4.13 (m, 8H), 4.04-4.03 (m, 2H), 3.96 (ddd, J = 16.3, 10.3, 3.5 Hz, 2H), 3.91-3.83 (m, 2H), 3.77-3.62 (m, 4H), 3.61- 3.55 (m, 1H), 3.50 (d, J = 9.6 Hz, 1H), 3.09 (t, J = 6.4 Hz, 2H), 2.00 (s, 3H), 1.98 (s, 3H), 1.96-1.91 (m, 2H). ¹³C NMR (101 MHz, Deuterium Oxide) δ 174.82, 173.03, 99.15, 98.35, 95.05, 93.90, 76.67, 73.68, 73.24, 72.06, |
| | | 70.91, 70.83, 70.52, 69.88, 69.62, |
| | | 69.12, 66.82, 66.56, 66.50, 64.65, |
| | | 63.24, 53.30, 53.15, 38.30, 26.05, |
| | | 22.19. HR-ESI-MS (m/z): [M − 3H] ³⁻ |
| | | calcd for C₃₁H₄₈N₃O₃₅S₄₃, 383.3656; |
| | | found, 383.3639. |
| 21 | 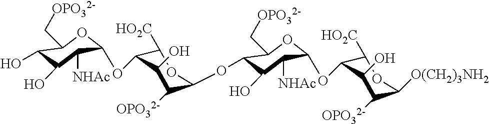 3-Aminopropyl-O-[(2-acetamido-6-0-phosphonato-2-deoxy-α-D-glucopyranosyl)- (1→4)-O-(2-O-phosphonato-α-L-idopyranosyluronate)-(1→4)-O-(2- acetamido-6-O-phosphonato-2-deoxy-α-D-glucopyranosyl)]-(1→4)-O-2-O- phosphonato-α-L-idopyranosiduronate | ¹H NMR (400 MHz, Deuterium Oxide) δ 5.13 (d, J = 10.1 Hz, 1H), 5.05 (dd, J = 10.2, 6.5 Hz, 2H), 4.79 (s, 1H), 4.15- 4.02 (m, 10H), 3.94 (td, J = 10.5, 3.4 Hz, 2H), 3.88-3.75 (m, 4H), 3.72- 3.62 (m, 4H), 3.59-3.57 (m, 1H), 3.51 (d, J = 9.2 Hz, 1H), 3.08 (t, J = 6.4 Hz, 1H), 2.02-1.90 (m, 8H). ¹³C NMR (151 MHz, Deuterium Oxide) δ 174.81, 172.75, 172.64, 100.08, 99.35, 95.88, 95.61, 77.05, 73.30, 73.24, 71.62, 71.33, 70.66, 69.65, 69.12, 66.86, 66.79, 66.61, 63.79, 53.58, |
| | | 53.34, 38.27, 26.07, 22.21, 22.15. ³¹p |
| | | NMR (162 MHz, Deuterium Oxide) δ |
| | | 0.75, 0.15, −0.10, −0.27. HR-ESI-MS |
| | | (m/z): [M − 2H]²⁻ calcd for |
| | | C₃₁H₅₃N₃O₃₅P₄₂, 575.5710; found, |
| | | 575.5759 |
| 22 | 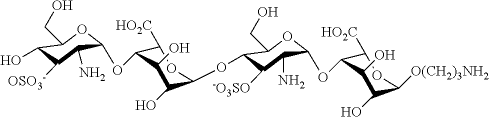 | ¹H NMR (600 MHz, Deuterium Oxide) δ 5.51 (d, J = 3.5 Hz, 1H), 5.41-5.39 (m, 1H), 4.95 (d, J = 2.8 Hz, 1H), 4.87 (d, J = 4.0 Hz, 1H), 4.56-4.50 (m, 1H), 4.48-4.42 (m, 2H), 4.19 (t, J = 3.6 Hz, 1H), 4.08-4.05 (m, 1H), 3.99 (t, J = 3.8 Hz, 1H), 3.93 (t, J = 9.6 Hz, 1H), 3.89 (s, 1H), 3.88-3.84 (m, 2H), |
| | 3-aminopropyl-O-[(2-amino-3-O-sulfonato- | 3.82-3.77 (m, 6H), 3.69-3.67 (m, |
| | 2-deoxy-α-D-glucopyranosyl)-(1→4)-O-(a- | 1H), 3.64-3.59 (m, 4H), 3.55 (dd, J = |
| | L-idopyranosyluronate)-(1→4)-O-(2-amino- | 10.4, 3.6 Hz, 1H), 3.06-3.04 (m, 2H), |
| | 3-O-sulfonato-2-deoxy-α-D- | 1.96-1.87 (m, 2H). ¹³C NMR (151 |
| | glucopyranosyl)]-(1→4)-O-α-L- | MHz, Deuterium Oxide) δ 174.57, |
| | idopyranosiduronate | 100.92, 100.59, 92.63, 92.42, 77.31, |
| | | 74.86, 74.78, 73.72, 72.07, 71.46, |
| | | 71.42, 69.98, 69.80, 69.50, 68.57, |
| | | 68.15, 67.39, 67.29, 66.43, 59.78, |
| | | 59.17, 53.63, 53.12, 38.06. HR-ESI- |
| | | MS (m/z): [M − 2H] ²⁻ calcd for |
| | | C₂₇H₄₅N₃O₂₇S₂₂⁻, 453.5846; found, |
| | | 453.5826 |
| 23 | 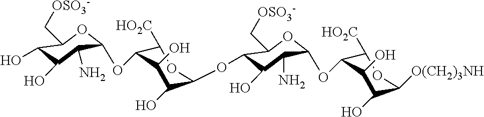 3-Aminopropyl-O-[(2-amino-6-O-sulfonato-2-deoxy-α-D-glucopyranosyl)-(1→4)-O-(α- L-idopyranosyluronate)-(1→4)-O-(2-amino-6-O-sulfonato-2-deoxy-α-D- glucopyranosyl)]-(1→4)-0-α-L-idopyranosiduronate | ¹H NMR (600 MHz, Deuterium Oxide) δ 5.41 (d, J = 3.7 Hz, 1H), 5.39 (d, J = 3.6 Hz, 1H), 5.04-5.03 (m, 3H), 4.15- 4.11 (m, 1H), 4.07-3.97 (m, 1H), 3.85-3.81 (m, 2H), 3.80-3.72 (m, 5H), 3.70-3.59 (m, 5H), 3.50-3.39 (m, 2H), 3.30 (td, J = 10.7, 10.0, 3.6 Hz, 1H), 3.08-3.05 (m, 2H), 2.76 (d, J = 5.2 Hz, 1H), 1.96-1.88 (m, 2H). ¹³C NMR (151 MHz, Deuterium Oxide) δ 171.27, 101.97, 100.86, 93.16, 92.70, |
| | | 76.71, 73.37, 73.00, 72.55, 71.93, |
| | | 69.35, 69.02, 68.46, 68.01, 67.80, |
| | | 67.35, 66.81, 65.95, 60.01, 59.35, |
| | | 54.03, 53.83, 38.01, 26.26. HR-ESI- |
| | | MS (m/z): [M − 2H] ²⁻ calcd for |
| | | C₂₇H₄₅N₃O₂₇S₂₂, 453.5846; found, |
| | | 453.5981 |
| 24 | 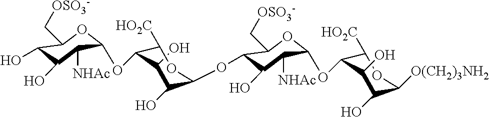 3-Aminopropyl-O-[(2-acetamido-6-0-sulfonato-2-deoxy-α-D-glucopyranosyl)- (1→4)-O-(α-L-idopyranosyluronate)-(1→4)-O-(2-acetamido-6-0-sulfonato-2-deoxy-α-D- glucopyranosyl)]-(1→4)-O-α-L-idopyranosiduronate | ¹H NMR (400 MHz, Deuterium Oxide) δ 4.99 (t, J = 4.2 Hz, 2H), 4.95 (d, J = 2.4 Hz, 1H), 4.87 (s, 1H), 4.77 (s, 1H), 4.60 (d, J = 1.9 Hz, 1H), 4.16 (ddd, J = 11.0, 8.0, 3.3 Hz, 2H), 4.06 (ddd, J = 10.9, 6.7, 1.8 Hz, 2H), 3.94 (dt, J = 4.8, 2.8 Hz, 2H), 3.89-3.72 (m, 6H), 3.64-3.62 (m, 2H), 3.60-3.53 (m, 4H), 3.50 (d, J = 10.4 Hz, 1H), 3.42 (d, J = 9.7 Hz, 1H), 2.99 (t, J = 6.6 Hz, 2H), 1.88-1.80 (m, 8H). ¹³C NMR |
| | | (101 MHz, Deuterium Oxide) δ |
| | | 174.43, 174.33, 173.41, 101.86, |
| | | 100.50, 95.24, 94.96, 77.25, 73.66, |
| | | 73.34, 71.03, 70.60, 69.91, 69.54, |
| | | 68.99, 68.58, 68.06, 67.55, 67.42, |
| | | 67.15, 66.67, 66.58, 66.53, 66.13, |
| | | 53.49, 53.22, 38.18, 26.21, 21.96. HR- |
| | | ESI-MS (m/z): [M − 2H]²⁻ calcd for |
| | | C₃₁H₄₉N₃O₂₉S₂₂, 495.5952; found, |
| | | 495.5974. |
| 25 | 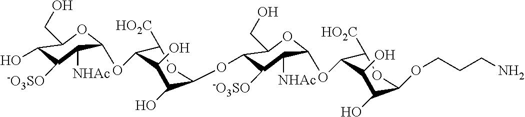 3-aminopropyl-O-[(2-acetamido-3-O-sulfonato-2-deoxy-α-D-glucopyranosyl)-(1→4)-O-(α-L- idopyranosyluronate)-(1→4)-O-(2-acetamido-3-O-sulfonato-2-deoxy-α-D-glucopyranosyl)]- (1→4)-O-(α-L-idopyranosyluronate)]. | ¹H NMR (400 MHz, Deuterium Oxide) δ 5.16 (d, J = 3.7 Hz, 1H), 5.08 (d, J = 3.8 Hz, 1H), 4.91 (s, 1H), 4.83 (d, J = 2.7 Hz, 2H), 4.42 (d, J = 2.5 Hz, 1H), 4.35 (d, J = 13.7 Hz, 2H), 4.07 (s, 2H), 4.03 (dd, J = 10.5, 3.6 Hz, 1H), 3.98- 3.89 (m, 4H), 3.87-3.75 (m, 10H), 3.67-3.62 (m, 2H), 3.61-3.52 (m, 3H), 3.10-3.04 (m, 2H), 1.92 (s, 3H), 1.90 (s, 3H). ¹³C NMR (151 MHz, Deuterium Oxide) δ 175.03, 174.40, 174.26, 100.30, 94.95, 94.63, 79.95, |
| | | 76.97, 74.96, 73.80, 71.81, 71.63, |
| | | 70.07, 69.84, 68.49, 68.32, 68.10, |
| | | 67.21, 66.42, 60.02, 59.57, 52.65, |
| | | 52.03, 38.15, 26.26, 22.09, 21.98. HR- |
| | | ESI-MS (m/z): [M − 2H]²⁻ calcd for |
| | | C₃₁H₄₉N₃O₂₉S₂₂, 495.5952; found, |
| | | 495.5972. |
| 26 | 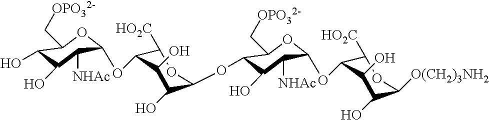 3-Aminopropyl-O-[(2-acetamido-6-0-phosphonato-2-deoxy-α-D-glucopyranosyl)- (1→4)-O-(α-L-idopyranosyluronate)-(1→4)-O-(2-acetamido-6-0-phosphonato-2-deoxy- α-D-glucopyranosyl)]-(1→4)-O-α-L-idopyranosiduronate | ¹H NMR (400 MHz, Deuterium Oxide) δ 4.98 (d, J = 3.1 Hz, 2H), 4.89 (s, 1H), 4.78 (s, 1H), 3.99-3.70 (m, 6H), 3.85 (ddd, J = 14.1, 8.1, 3.2 Hz, 4H), 3.80-3.73 (m, 1H), 3.69-3.49 (m, 9H), 3.48-3.40 (m, 1H), 2.99 (t, J = 6.4 Hz, 2H), 1.92-1.82 (m, 8H). ¹³C NMR (151 MHz, Deuterium Oxide) δ 174.65, 174.46, 174.34, 101.85, 100.44, 95.06, 94.58, 81.76, 77.10, 74.05, 73.37, 71.07, 70.33, 70.28, 69.87, 69.15, 68.97, 68.52, 68.46, |
| | | 68.13, 67.97, 67.11, 63.64, 63.30, |
| | | 53.64, 53.43, 38.24, 26.31, 21.97. 31p |
| | | NMR (162 MHz, D₂O) 8 1.35, 0.85. |
| | | HR-ESI-MS (m/z): [M − 2H]²⁻ calcd for |
| | | C₃₁H₅₃N3O29P22, 495.6047; found, |
| | | 495.6060. |
| 27 | 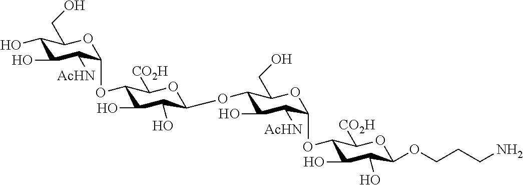 | ¹H NMR (400 MHz, Deuterium Oxide) δ 5.29 (dd, J = 5.4, 3.8 Hz, 2H), 4.46 (dd, J = 22.1, 8.0 Hz, 2H), 3.95 (dd, J = 24.7, 9.5 Hz, 3H), 3.82 (dd, J = 10.8, 3.8 Hz, 3H), 3.77-3.59 (m, 13H), 3.56-3.51 (m, 1H), 3.45 (d, J = 9.2 Hz, 1H), 3.32-3.23 (m, 2H), 3.06 (t, J = 6.9 Hz, 2H), 1.96 (d, J = 3.6 Hz, 5H), 1.91 (s, 2H). ¹³C NMR (101 MHz, Deuterium Oxide) 8 174.90, 168.16, 102.73, 102.12, 97.54, 77.89, 76.42, |
| | | 75.73, 75.47, 74.86, 73.65, 73.04, |
| | 3-Aminopropyl-O-[(2-deoxy-2-acetamido-α- | 71.49, 69.32, 68.72, 60.41, 58.94, |
| | D-glucopyranosyl)-(1→4)-O-(B-D- | 53.05, 37.73, 26.13, 22.41. HRMS: |
| | glucopyranosyluronic acid)-(1→4)-O-(2- | m/z: calculated for C₃₁H₅₁N₃O₂₃; |
| | deoxy-2-acetamido-α-D-glucopyranosyl)- | 833.2913; found: 833.2910. |
| | (1→4)-O-(B-D-glucopyranosyluronic acid)]. | |
| 28 | 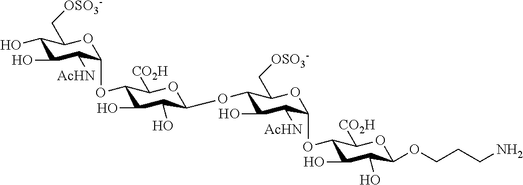 3-Aminopropyl-O-[(2-deoxy-2-acetamido-6-O-sulfonate-α-D-glucopyranosyl)-(1→4)-O- (B-D-glucopyranosyluronic acid)-(1→4)-O-(2-deoxy-2-acetamido-6-O-sulfonate-α-D- glucopyranosyl)-(1→4)-O-(B-D-glucopyranosyluronic acid)]. | ¹H NMR (400 MHz, Deuterium Oxide) δ 5.28 (t, J = 3.2 Hz, 2H), 4.55 (d, J = 7.9 Hz, 1H), 4.44 (d, J = 8.0 Hz, 1H), 4.35 (s, 1H), 4.24 (dd, J = 11.0, 2.8 Hz, 1H), 4.11 (t, J = 11.4 Hz, 2H), 3.98 (d, J = 9.5 Hz, 1H), 3.91 (d, J = 9.3 Hz, 1H), 3.87 (d, J = 5.5 Hz, 1H), 3.85 (d, J = 5.4 Hz, 2H), 3.76 (d, J = 9.7 Hz, 3H), 3.71 (m, 3H), 3.66 (d, J = 5.2 Hz, 2H), 3.62 (d, J = 9.6 Hz, 2H), 3.49 (t, J = 9.6 Hz, 1H), 3.28 (q, J = 8.4 Hz, 2H), 3.07 (t, J = 6.8 Hz, 2H), 1.97 (s, 3H), 1.96 (s, 3H), 1.94-1.90 (m, 2H). ¹³C NMR (101 MHz, Deuterium Oxide) δ 174.34, 173.02, 102.22, 101.92, 97.67, 97.33, 77.44, 76.74, |
| | | 76.65, 76.03, 75.80, 74.99, 74.58, |
| | | 73.26, 73.17, 70.40, 70.36, 69.05, |
| | | 69.03, 68.79, 67.99, 66.34, 65.71, |
| | | 53.41, 53.12, 37.56, 26.66, 21.92. |
| | | HRMS: m/z: calculated for |
| | | C₃₁H₄₉N₃O₂₉S₂²⁻; 495.5952; found: |
| | | 495.5910. |
| 29 | 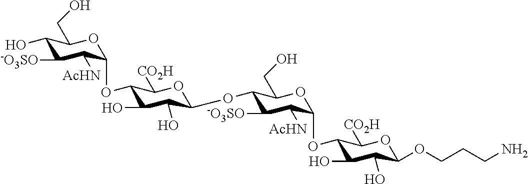 3-Aminopropyl-O-[(2-deoxy-2-acetamido-3-O-sulfonate-α-D-glucopyranosyl)-(1→4)-O- (B-D-glucopyranosyluronic acid)-(1→4)-O-(2-deoxy-2-acetamido-3-O-sulfonate-α-D- glucopyranosyl)-(1→4)-O-(ß-D-glucopyranosyluronic acid)]. | ¹H NMR (400 MHz, Deuterium Oxide) δ 5.39 (t, J = 4.1 Hz, 2H), 4.62 (d, J = 7.8 Hz, 1H), 4.56 (d, J = 9.7 Hz, 1H), 4.51 (d, J = 8.2 Hz, 1H), 4.48-4.42 (m, 1H), 4.09 (ddd, J = 14.1, 10.7, 3.6 Hz, 2H), 4.03-3.95 (m, 3H), 3.93 (d, J = 2.6 Hz, 1H), 3.89-3.80 (m, 7H), 3.75 (q, J = 7.6, 6.4 Hz, 4H), 3.69 (d, J = 8.9 Hz, 1H), 3.42 (t, J = 8.5 Hz, 1H), 3.33 (s, 1H), 3.16 (t, J = 6.6 Hz, 2H), 2.03 (s, 3H), 2.02 (s, 3H), 1.99 (d, J = 6.2 Hz, 2H). ¹³C NMR (101 MHz, Deuterium Oxide) δ 176.90, 176.86, 104.72, 103.73, 100.24, 99.92, 82.14, 79.49, 79.31, 79.14, 78.63, 78.37, 75.86, 75.81, 75.66, 74.50, 74.02, |
| | | 70.61, 70.47, 62.35, 61.89, 55.22, |
| | | 54.73, 40.08, 29.21, 24.68, 24.61. |
| | | HRMS: m/z: calculated for |
| | | C₃₁H₄₉N₃O₂₉S₂²⁻; 495.5952; found: |
| | | 495.5919. |
| 30 | 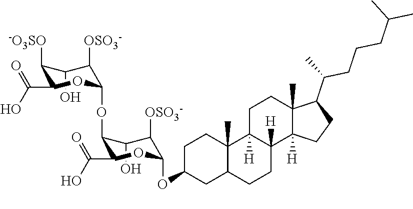 | ¹H NMR (400 MHz, Deuterium Oxide) δ 5.15 (dd, J = 35.1, 23.4 Hz, 2H), 4.88 (s, 1H), 4.47 (d, J = 17.9 Hz, 1H), 4.19- 3.95 (m, 4H), 3.57 (t, J = 6.1 Hz, 1H), 3.00 (q, J = 7.0 Hz, 2H), 1.88 (dd, J = 11.3, 5.5 Hz, 1H), 1.77-1.53 (m, 5H), 1.49-1.38 (m, 3H), 1.32-1.15 (m, 11H), 1.09-0.90 (m, 9H), 0.84- 0.73 (m, 14H), 0.58 (s, 3H). HRMS m/z calculated for C₃₉H₆₁O₂₂S₃³⁻ :977.2833 found (m/z + 3H): 326.7687 |
| | Cholesteryl-O-((2,4-O-disulfonato)-α-L- | |
| | idopyranosyl uronic acide-α(1→4) (2-0- | |
| | sulfonato))-α-L-idopyranoside uronic acide | |
| 31 | 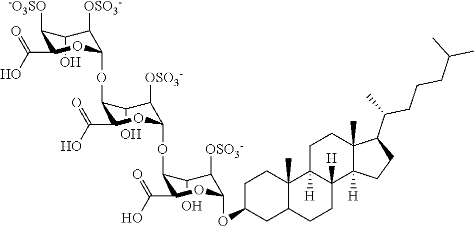 | ¹H NMR (400 MHz, Deuterium Oxide) δ 5.14 (dd, J = 21.1, 11.3 Hz, 3H), 4.87 (d, J = 8.9 Hz, 1H), 4.58 (s, 1H), 4.40 (s, 1H), 4.04 (dd, J = 39.0, 26.3 Hz, 6H), 3.65-3.53 (m, 1H), 2.98 (d, J = 7.7 Hz, 2H), 1.92-1.87 (m, 1H), 1.75- 1.55 (m, 5H), 1.50-1.38 (m, 2H), 1.34-1.15 (m, 11H), 1.10-0.90 (m, 9H), 0.83-0.72 (m, 12H), 0.58 (s, 3H). HRMS m/z calculated for C₄₅H₇₀O₃₁S₄₄-: 1234.2806 found (m/z + 4H): 308.5698. |
| | Cholesteryl-O-((2,4-O-disulfonato-3-O- | |
| | benzyl-)-α-L-idopyranosyl uronate- | |
| | α(1→4)(2-O-sulfonato-3-O-benzyl)-α-L- | |
| | idopyranosyl uronate-a(1→4)(2-0- | |
| | sulfonato-3-O-benzyl))-α-L-idopyranoside | |
| | uronate | |
| 32 | 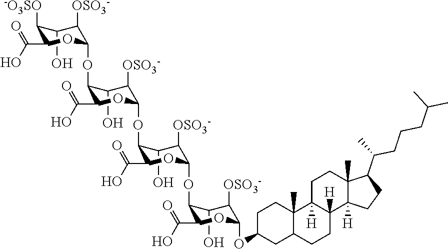 | ¹H NMR (400 MHz, Deuterium Oxide) δ 4.96 (t, J = 12.1 Hz, 4H), 4.65 (s, 2H), 4.43-4.26 (m, 2H), 4.00-3.81 (m, 9H), 3.49-3.38 (d, J = 22.3 Hz, 1H), 2.85-2.79 (m, 2H), 1.73 (d, J = 12.0 Hz, 1H), 1.62-1.39 (s, 5H), 1.36- 1.22 (m, 4H), 1.17-0.96 (m, 13H), 0.94-0.75 (m, 8H), 0.86-0.57 (m, 12H), 0.43 (s, 3H). HRMS m/z calculated for C₅₁H₈₀O₄₀S₅⁵⁻ :1492.2857; found (m/z + 5H):298.4568. |
| | Cholesteryl-O-((2,4-O-disulfonato)-α-L- | |
| | idopyranosyl uronate-a(1-+4) (2-0- | |
| | sulfonato)-α-L-idopyranosyl uronate- | |
| | α(1→4) (2-Osulfonato)-α-L-idopyranosyl | |
| | uronate-α(1→4) (2-Osulfonato))-α-L- | |
| | idopyranoside urinate | |
| 33 | 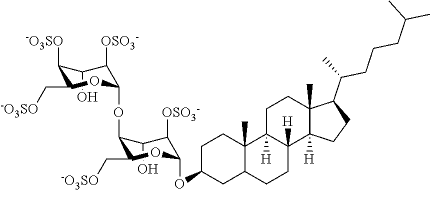 Cholestanyl-O-((2,4,6-O-trisulfonato)-α-L- idopyranosyl-α(1→4) (2,6-O-disulfonato))-α-L-idopyranoside | ¹H NMR (600 MHz, Deuterium Oxide) δ 5.17 (d, J = 40.7 Hz, 2H), 4.55 (d, J = 8.0 Hz, 1H), 4.37-4.31 (m, 3H), 4.24 (dt, J = 10.9, 6.8 Hz, 6H), 3.85 (s, 1H), 3.73 (s, 1H), 1.96 (d, J = 12.4 Hz, 1H), 1.80 (s,1H), 1.86 (s, 1H), 1.72 (s, 1H), 1.66 (s, 2H), 1.51 (td, J = 13.1, 7.3 Hz, 4H), 1.41-1.21 (m, 10H), 1.10 (dd, J = 14.2, 5.1 Hz, 6H), 1.00 (h, J = 12.5, 12.0 Hz, 4H), 0.90 (d, J = 6.6 Hz, 3H), 0.83 (dd, J = 6.6, 2.6 Hz, 6H), 0.80 (s, 3H), 0.65 (s, 3H). ¹³C NMR (151 MHz, Methanol-d₄) δ 99.82, 96.78, 77.24 (d, J = 5.5 Hz), 74.51, 72.90, 68.08, 67.03, 66.94, 66.55, 65.00, 64.80, 56.52, 56.26, |
| | | 54.29, 44.63, 42.35, 40.00, 39.24, |
| | | 36.82, 35.92, 35.69, 35.48, 35.31, |
| | | 33.51, 31.91, 29.55, 28.61, 28.56, |
| | | 27.97, 27.70, 23.86, 23.46, 21.85, |
| | | 21.59, 20.93, 17.82, 11.14. |
| 34 | 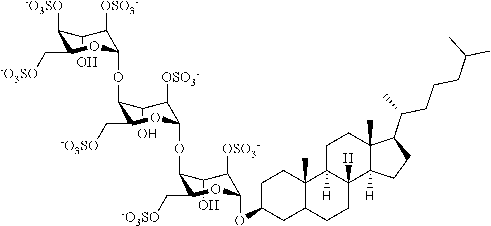 | ¹H NMR (400 MHz, Deuterium Oxide) δ 5.40 (d, J = 11.5 Hz, 1H), 5.18 (s, 2H), 5.00-4.85 (m, 2H), 4.60-4.41 (m, 6H), 4.35-4.13 (m, 3H), 4.05 (d, J = 15.4 Hz, 3H), 3.98-3.72 (m, 3H), 2.24 (bs, 1H), 2.16-1.90 (m, 5H), 1.83-1.73 (m, 4H), 1.62-1.50 (m, 9H), 1.40-1.25 (m, 10H), 1.19-1.18 (m, 4H), 1.13 (d, J = 6.0 Hz, 6H), 1.07 (s, 3H), 0.93 (s, 3H). HRMS m/z calculated for C₄₅H₇₁O₃₇S₇⁷⁻: 1427.1758 found (m/z + 7H): 204.8989. |
| | Cholestanyl-O-((2,4,6-O-trisulfonato)-α-L- | |
| | idopyranosyl-α(1→4)(2,6-O-disulfonato)-α- | |
| | L-idopyranosyl-α(1→4) (2,6-O- | |
| | disulfonato))-α-L-idopyranoside | |
| 35 | 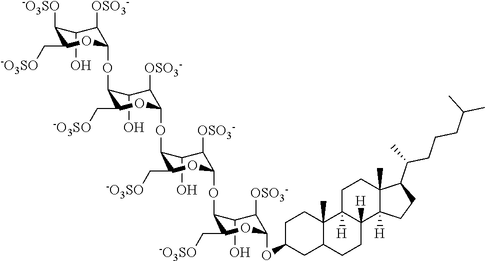 | ¹H NMR (400 MHz, Deuterium Oxide) δ 5.20-5.12 (m, 1H), 5.01-4.88 (m, 3H), 4.70-4.58 (m, 2H), 4.53-4.41 (m, 2H), 4.29 (s, 8H), 4.16-4.06 (m, 2H), 4.01-3.90 (m, 3H), 3.88-3.80 (m, 3H), 3.78-3.68 (m, 3H), 3.58- 3.48 (m, 1H), 2.05-1.84 (m, 3H), 1.78- 1.52 (m, 7H), 1.48-1.28 (m, 8H), 1.22-1.10 (m, 6H), 1.06-0.82 (m, 16H), 0.71 (s, 3H). HRMS m/z calculated for C₅₁H₇₉O₄₈S₉⁹⁻: 1747.1277; found (m/z + 9H): 195.1329. |
| | Cholesteryl-O-((2,4,6-O-trisulfonato)-α-L- | |
| | idopyrnosyl-α(1→4)(2,6-O-disulfonato-3-0- | |
| | benzyl 3)-α-L-idopyrnosyl-α(1→4) (2,6-O- | |
| | disulfonato)-α-L-idopyrnosyl-α(1→4) (2,6- | |
| | O-disulfonato))-α-L-idopyranose | |

### Biological Studies

Microarray Studies: The compounds of the present invention were covalently immobilized on epoxy group collated glass slides. Slides were rehydrated with dH₂O and incubated for 30 min in a staining dish with 50° C. prewarmed ethanolamine (0.05 M) in Tris-HCl (0.1 M, pH 9.0) to block the remaining reactive epoxy groups on the slide surface, then washed with 50° C. prewarmed dH₂O. Slides were centrifuged at 200 g for 3 min, then fitted with the ProPlate Multi-Array 16-well slide module (Grace Bio-lab P37001) to divide into the subarrays (blocks). Slides were washed with PBST (0.1% Tween 20), aspirated, and blocked with 200 μL/subarray of blocking buffer (PBS pH 7.3+1% w/v ovalbumin) for 1 h at RT with gentle shaking. Next, the blocking solution was aspirated and 100 μL/block of primary chemokines diluted in PBS pH 7.3+1% w/v ovalbumin were incubated with gentle shaking for 2 hours at RT. Slides were washed 4 times with PBST, then with PBS (without Tween-20) for 2 min. Bound antibodies were detected by incubating with biotinylated secondary detections (1 ng/μL) diluted in PBS, 200 μL/block at RT for 1 h. Slides were washed 4 times with PBST, then with PBS (without Tween-20) for 2 min and biotinylated antibodies were detected with Cy3-SA (1.2 μg/mL). Slides were washed 4 times with PBST, then with PBS for 10 min followed by removal from the ProPlate Multi-Array slide module and immediately dipping in a staining dish with dH₂O for 10 min with shaking. The slides were then centrifuged at 200 g for 3 min, and the dry slides immediately scanned. Binding was tested at three serial dilutions, then detected with the relevant biotinylated secondary antibody (1 μg/mL) followed by Cy3-strepavidin (1.5 μg/mL). Arrays were scanned and RFUs were calculated of chemokines binding to 100 μM glycans printed at four replicates. Rank binding of chemokines (each at three dilutions) to glycans printed at four replicates each was calculated and summarized in Table 1. For each binding assay per printed block, the maximum RFU was determined and set as 100% binding. Then, binding to all other glycans in the same block was ranked in comparison to the maximal binding, and average rank binding (and SEM) for each glycan across the three examined concentrations of each chemokine was calculated. This analysis allowed to compare the glycan-binding profiles of the different chemokines and dissect their binding preferences.

Table 1. Chemokine glycan microarray binding assay. Binding was tested at 3 serial dilutions, then detected with the relevant biotinylated secondary antibody (1 μg/ml) followed by Cy3-Strepavidin (1.5 μg/ml) (Table SX). Arrays were scanned, relative fluorescent units (RFU) obtained, and maximum RFU determined and set as 100% binding. Then rank binding (per printed glycan per concentration, per each chemokine dilution, per printed block) was determined. Since each glycans was printed at 2 concentration, 100% binding was set separately for each concentration. Then, binding to all the other glycans at the same concentration was ranked in comparison to the maximal binding, and the average rank binding and SEM for each glycan across the two glycan concentrations and three examined dilutions of each chemokine was calculated (n=6, 2 glycan concentrations across 3 chemokine dilutions). This analysis allowed to compare the glycan binding profiles of the different chemokines and dissect their binding preferences. The mean rank is shown as a heatmap of all the examined binding assays together (red highest, blue lowest and white 50^{th} percentile of ranking).

| ID | CCL13 | CCL21 | CXCL12 | CCL28 | CCL7 | CXCL10 | CXCL8 | CCL5 | CCL2 | |
|---|---|---|---|---|---|---|---|---|---|---|
| 1 | 74 | 52 | 62 | 46 | 30 | 47 | 17 | 2 | 4 | |
| 2 | 82 | 63 | 66 | 58 | 30 | 45 | 12 | 2 | 2 | |
| 3 | 69 | 46 | 38 | 72 | 17 | 24 | 5 | 1 | 0 | |
| 4 | 67 | 48 | 56 | 56 | 33 | 44 | 12 | 2 | 2 | |
| 5 | 81 | 62 | 64 | 80 | 58 | 57 | 62 | 15 | 23 | |
| 6 | 94 | 71 | 79 | 84 | 55 | 67 | 73 | 38 | 25 | |
| 7 | 86 | 83 | 92 | 80 | 75 | 78 | 63 | 30 | 36 | |
| 8 | 72 | 60 | 45 | 54 | 12 | 23 | 13 | 10 | 0 | |
| 9 | 53 | 79 | 75 | 51 | 94 | 82 | 71 | 75 | 66 | |
| 10 | 88 | 86 | 98 | 76 | 87 | 96 | 83 | 86 | 69 | |
| 11 | 67 | 80 | 82 | 84 | 79 | 74 | 95 | 80 | 67 | |
| 12 | 70 | 50 | 91 | 72 | 80 | 87 | 96 | 91 | 88 | |
| 13 | 70 | 69 | 69 | 43 | 56 | 52 | 37 | 31 | 13 | |
| 14 | 21 | 23 | 11 | 21 | 1 | 11 | 2 | 1 | 0 | |
| 15 | 64 | 62 | 51 | 36 | 45 | 39 | 13 | 3 | 3 | |
| 16 | 24 | 27 | 16 | 22 | 2 | 14 | 3 | 1 | 0 | |
| 17 | 69 | 73 | 80 | 54 | 89 | 96 | 94 | 82 | 95 | |
| 18 | 64 | 48 | 45 | 66 | 35 | 27 | 9 | 4 | 1 | |
| 19 | 74 | 66 | 79 | 79 | 91 | 89 | 79 | 95 | 80 | |
| 20 | 79 | 71 | 69 | 66 | 86 | 64 | 53 | 54 | 30 | % |
| 21 | 87 | 78 | 59 | 78 | 56 | 55 | 29 | 14 | 2 | 100 |
| 22 | 58 | 83 | 84 | 62 | 78 | 65 | 51 | 20 | 28 | 75 |
| 23 | 45 | 25 | 25 | 38 | 11 | 15 | 5 | 3 | 1 | 50 |
| 24 | 81 | 79 | 66 | 79 | 73 | 41 | 19 | 24 | 13 | 25 |
| 26 | 70 | 81 | 75 | 39 | 40 | 48 | 10 | 5 | 1 | 0 |

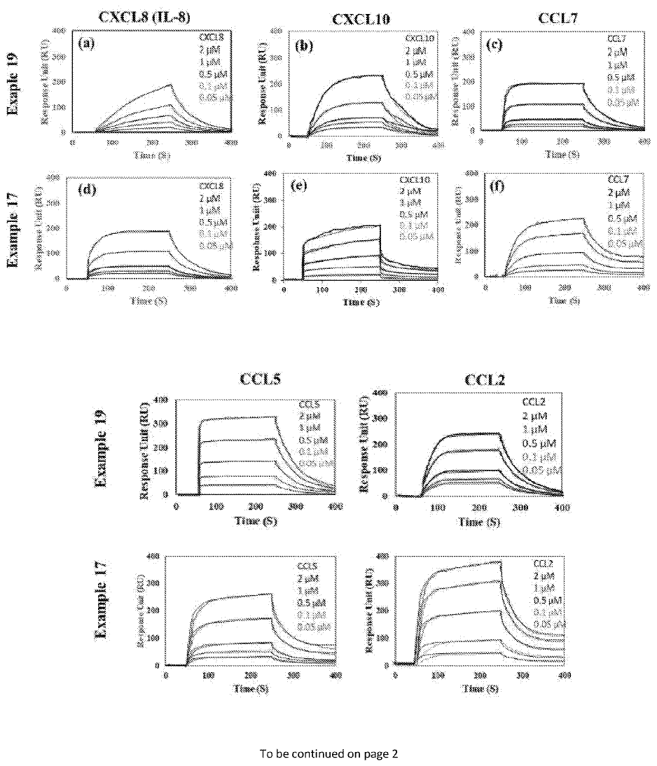

Surface Plasmon Resonance (SPR) binding kinetics The compound of present invention (test compound, Example 12) was covalently immobilized on sensor chip via coupling reaction. At first, the dextran matrix on CM5 chip was activated with NHS (0.02 M) and EDC coupling reagent (0.2 M) at a flow rate of 5 μL min⁻¹ for 15 min before activating with 50 μL of test compound (0.5 mM) in HBS-EP buffer. In the negative control cell after EDC and NHS activation, 0.5 mM of ethanol amine solution was flowed. The positive RU response on test compound confirmed immobilization of the HS ligand on CM5 chips. Then, different chemokines at a flow rate of 50 μL/min and 25° C. in HBS-EP buffer without growth factor was then flowed over the sensor surface for 3 min to enable association/dissociation. Kinetic analysis was performed using the BIA evaluation software for T100. Association and dissociation phase data were globally fitted to a simple 1:1 interaction model. The results are shown in the table 2 below. The results clearly suggest that compounds of the present invention have strong binding affinity to CCL2 chemokine. FIG. 1 shows the chemokine binding profile of compounds of present invention.

Table 2: SPR analysis of kinetic rate constants and equilibrium affinities of compounds of present invention binding to chemokines

| Com- | Chemo- | | | |
|---|---|---|---|---|
| pound | kine | K_{D}(μm) | Kₒₙ(M⁻¹S⁻¹) | K_{off}(S⁻¹) |
| Ex-12 | CXCL8 | 9.34 ± 0.21 | 3.16 ± 0.08 × 10⁵ | 2.96 ± 0.1 × 10⁻¹ |
| | CXCL10 | 15.34 ± 0.21 | 1.44 ± 0.46 × 10⁴ | 2.2 ± 0.38 × 10⁻¹ |
| | CXCL12 | 18.78 ± 0.44 | 1.1 ± 0.46 × 10⁴ | 2.1 ± 0.78 × 10⁻¹ |
| | CCL5 | 5.72 ± 0.24 | 4.2 ± 0.46 × 10⁴ | 2.4 ± 0.38 × 10⁻¹ |
| | CCL2 | 1.67 ± 0.3 | 1.26 ± 0.08 × 10⁵ | 2.1 ± 0.5 × 10⁻¹ |
| Ex-17 | CXCL8 | 11.67 ± 0.17 | 1.87 ± 0.22 × 10⁴ | 2.18 ± 0.28 × 10⁻¹ |
| | CXCL10 | 11.02 ± 0.37 | 1.53 ± 0.26 × 10⁴ | 1.69 ± 0.33 × 10⁻¹ |
| | CCL7 | 11.3 ± 0.18 | 2.53 ± 0.15 × 10⁴ | 2.86 ± 0.21 × 10⁻¹ |
| | CCL5 | 6.81 ± 0.33 | 4.37 ± 0.31 × 10⁴ | 2.98 ± 0.31 × 10⁻¹ |
| | CCL2 | 1.89 ± 0.24 | 11.17 ± 0.32 × 10⁴ | 2.11 ± 0.34 × 10⁻¹ |
| Ex-19 | CXCL8 | 15.93 ± 0.23 | 1.23 ± 0.17 × 10⁴ | 1.96 ± 0.29 × 10⁻¹ |
| | CXCL10 | 10.7 ± 0.37 | 2.07 ± 0.26 × 10⁴ | 2.24 ± 0.35 × 10⁻¹ |
| | CCL7 | 11.51 ± 0.17 | 1.96 ± 0.22 × 10⁴ | 2.25 ± 0.29 × 10⁻¹ |
| | CCL5 | 2.34 ± 0.44 | 9.15 ± 0.34 × 10⁴ | 2.14 ± 0.48 × 10⁻¹ |
| | CCL2 | 9.82 ± 0.18 | 2.07 ± 0.13 × 10⁴ | 2.03 ± 0.17 × 10⁻¹ |

The compounds of the present invention showed high affinity binding to CCL2 chemokines. The results of the studies suggested that the compounds of present invention are potential ligands that can modulate CCL2 chemokine activities.

### Surface Plasmon Resonance (SPR) Binding Kinetics

The compound of present invention (test compound, Example 12, 17 & 18) was covalently immobilized on sensor chip via coupling reaction. At first, the dextran matrix on CM5 chip was activated with NHS (0.02 M) and EDC coupling reagent (0.2 M) at a flow rate of 5 μL min⁻¹ for 15 min before activating with 50 μL of test compound (0.5 mM) in HBS-EP buffer. In the negative control cell after EDC and NHS activation, 0.5 mM of ethanol amine solution was flowed. The positive RU response on test compound confirmed immobilization of the HS ligand on CM5 chips. Then, different chemokines at a flow rate of 50 μL/min and 25° C. in HBS-EP buffer without growth factor was then flowed over the sensor surface for 3 min to enable association/dissociation. Kinetic analysis was performed using the BIA evaluation software for T100. Association and dissociation phase data were globally fitted to a simple 1:1 interaction model. The results are shown in the table below. The results clearly suggest that compounds of the present invention have strong binding affinity to CCL2 chemokine. FIG. 1 shows the chemokine binding profile of compounds of present invention.

### Cell Proliferation Assay

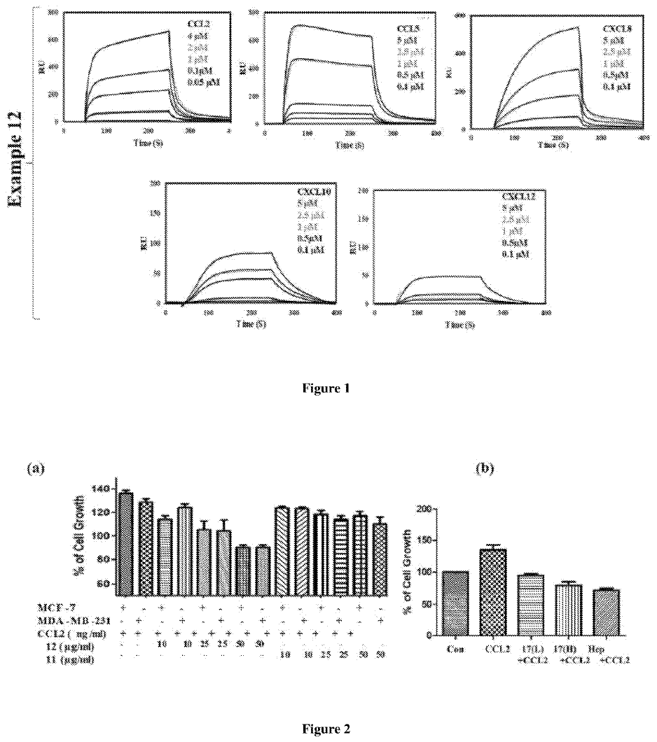

MCF-7 or MDA-MB-231 (approximately 10⁴) were plated on 96 well plates in a RPMI-1640 medium in 1% FBS without growth supplements. Cells were incubated for 4 hr before the experiments. First HS biomimetics, test compounds (11 and 12, 10 or 50 μg/ml) and native heparin (10 or 50 μg/ml) were preincubated with CCL2 (50 ng/ml) and added to the cells. After 48 h of incubation, cell were washed and fixed with paraformaldehyde. Cell were stained with 4% sulforhodamine B in 1% acetic acid for 30 min and washed with 1% acetic acid solution. Cell proliferation was determined with 2-(4-iodophenyl)-3-(4-nitrophenyl)-5-(2,4-disulfophenyl)-2H-tetrazolium monosodium salt at 450 nm. The results are shown in FIG. 2.

### Cell-Division Cycle Analysis

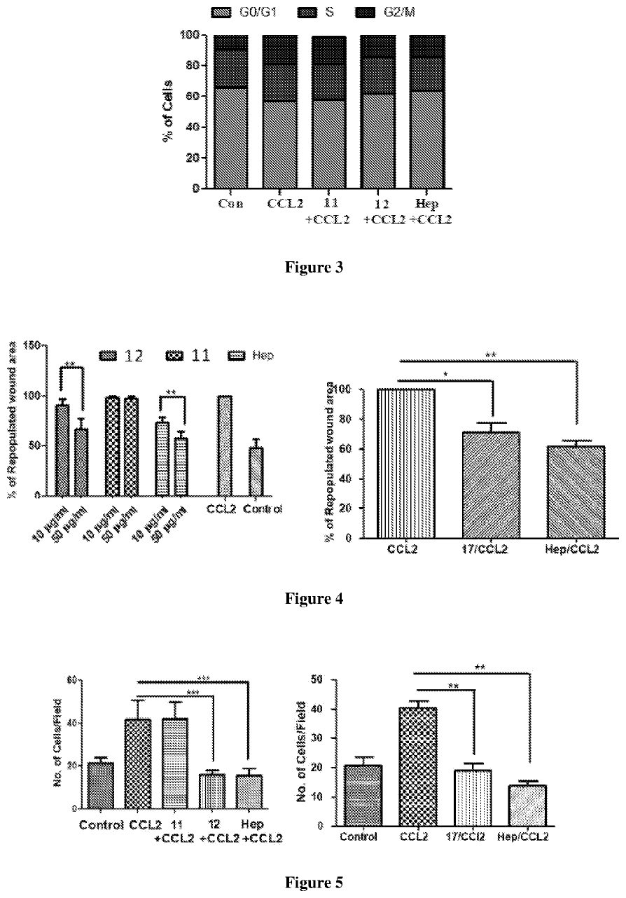

MCF-7 cells were treated with chemokines and HS mimics, test compounds as mentioned above after 72 h, cells were harvested and fixed overnight in 70% ethanol at −20° C. The fixed cells were incubated with propidium iodide (5 μg/mL) and RNase (10 μg/mL) for 30 min at 37° C. The stained cells were analyzed with a flow cytometer. The DNA content in the G0/G1, S and G2/M phases was quantified by using ModFitLT version 3.0 software. The results are shown in FIG. 3.

### Wound Healing Assay:

MCF-7 cells were cultured on 24-well plates in RPMI-1640 medium. After monolayer formation, cells were starved with serum free medium for 24 h. Then, wound was created by scratching the monolayer with 1000 μl pipette tip. Cells were treated with heparin and its mimics, test compounds (Example 12, 11, 10 or 50 μg/ml) with CCL2 (50 ng). After 8 h, CCL2 treated monolayer showed complete wound healing. At that point, % of cell migration distance of heparin mimics, test compounds treated cells were quantified. The results are shown in FIG. 4.

### Cell Invasion Assay:

Cell invasion assay was performed in 24-well boyden chamber inserts with 8 μM pore. Upper chamber of transwell inserts were coated with Matrigel. The bottom chamber contained 600 μL RPMI-1640 medium supplemented with 1% FBS and CCL2 (50 ng/ml) with heparin mimics, test compounds (12, 11 and heparin). MCF-7 cells were added to the upper chamber. After 24 h incubation at 37° C., non-invading cells were removed and cells migrated through the membrane to the lower surface were fixed and stained with 0.5% crystal violet for 30 min and quantified by bright field imaging. The results are shown in FIG. 5.

### Western Blot Analysis:

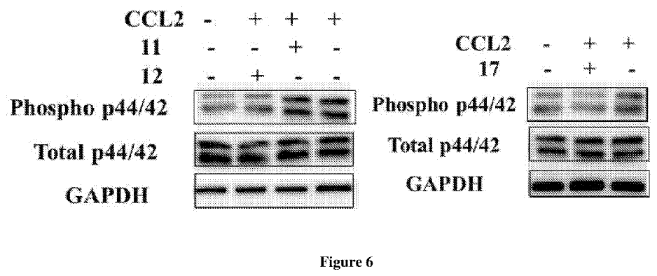

MCF-7 cells were grown in 100 mm Petri dishes and treated with CCL2 (50 ng) and heparin mimics (50 μg) for half hr. The cells were pelleted, and treated with protease inhibitors before treating with lysis buffer containing 150 mM NaCl, 1% NP-40, 0.25% sodium dodecyl sulfate (SDS), 1 mM ethylenediaminetetraacetic acid (EDTA), and 1 mM phenylmethane sulfonyl fluoride (PMSF) in 50 mM Tris-Cl (pH 7.4). After 1 h, the supernatant was collected by centrifugation (14000 rpm) for 15 min and stored in aliquots. The protein content was quantified using the Bradford method. The protein (35 μg) was loaded on SDS-polyacrylamide gel electrophoresis (10%) and transferred onto a polyvinylidene fluoride (PVDF) membrane. The membrane was incubated for 2 h with specific antibodies corresponding to MAPK. The membranes were incubated with horse radish peroxidase (HRP)-conjugated secondary antibody for 1 h at room temperature, and visualization was done using an Immobilon Western Chemiluminescent HRP substrate kit (Millipore Corporation, MA, USA) with GAPDH as internal standard. BioRad's Protein Ladder (Thermo, EU) was used to determine the molecular weights of the protein bands. The results are shown in FIG. 6.

Western blot analysis of p42/44 showed that MCF-7 cells treated with the compounds of present invention expressed low level of MAPK compared. Overall, these results suggest that the compounds of present invention are potential ligand to modulate CCL2 activity and anticancer therapy.

The foregoing examples are merely illustrative and are not to be taken as limitations upon the scope of the invention. Various changes and modifications to the disclosed embodiments will be apparent to those skilled in the art. Such changes and modifications may be made without departing from the scope of the invention.

1. A heparin sulfate compound of formula (I) or a stereoisomer, a tautomer, a conformer, a pharmaceutically acceptable salt or a pharmaceutically acceptable solvate thereof; 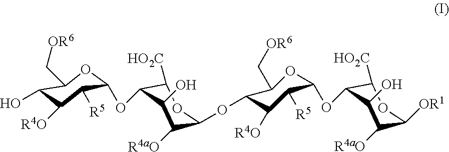
wherein:
R¹ is —(CH₂)₃NH₂; R⁴ is H or S0₃;
R^{4a} is H or S0₃;
R⁵ is NH₂ or NHCOCH3; and R⁶ is H, SO3 and PO4²⁻.

2. The heparin sulfate compound as claimed in claim 1, wherein the compound is selected from the compound of formula (1a) or a stereoisomer, a tautomer, a conformer, a pharmaceutically acceptable salt or a pharmaceutically acceptable solvate thereof; 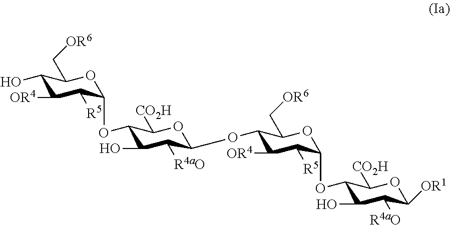
wherein: R¹ is —(CH₂)₃NH2;
R⁴ is H or S0₃;
R^{4a} is H or S0₃;
R⁵ is NH₂ or NHCOCH3; and R⁶ is H or SO3.

3. A heparin sulfate compound of formula (II) or a stereoisomer, a tautomer, a conformer, a pharmaceutically acceptable salt, a homooligosaccharides or a pharmaceutically acceptable solvate thereof; 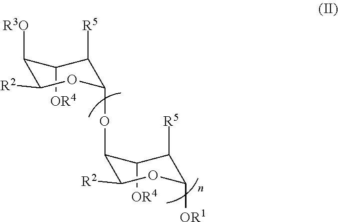
wherein:
R¹ 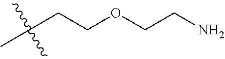 [SMILES: *CCOCCN]
or cholestenol;
R² independently represents COOH or CH₂OH;
R³ independently represents H or SO₃⁻;
R⁴ independently represents H, SO₃⁻;
R⁵ independently represents OH, OSO₃⁻,
n is 0, 1, 2, or 3.

4. The heparin sulfate compound as claimed in claim 3, wherein:
R¹ is 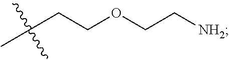 [SMILES: *CCOCCN]
R² independently represents COOH;
R³ independently represents H or SO₃⁻;
R⁴ independently represents H, or SO₃⁻;
R⁵ independently represents OH, or OSO₃⁻; and
n is 0, 1, 2 or 3.

5. The heparin sulfate compound as claimed in claim 3, wherein:
R¹ is cholestenol;
R² independently represents COOH;
R³ independently represents SO3;
R⁴ independently represents H;
R⁵ independently represents OSO3; and n is 1, 2 or 3.

6. The heparin sulfate compound as claimed in claim 3, wherein:
R¹ is cholestenol
R² independently represents CH₂OH;
R³ independently represents SO3;
R⁴ independently represents H;
R⁵ independently represents OSO3; and n is 1, 2, or 3.

7. The compound as claimed in claim 1,
wherein the compound is selected from the group consisting of:
Ethoxy-2-aminoethoxyl-O-α-L-idopyranoside Uronic Acid;
Ethoxy-2-aminoethoxyl-O-(4-O-sulfonato)-α-L-idopyranoside Uronic Acid;
Ethoxy-2-aminoethoxyl-O-(2,4-O-disulfonato)-α-L-idopyranoside Uronic Acid;
Ethoxy-2-aminoethoxyl-O-(α-L-idopyranosyl Uronic Acid-α(1→4))-α-L-idopyranosyl Uronic Acid;
Ethoxy-2-aminoethoxyl-O-((4-O-sulfonato)-α-L-idopyranosyl Uronic Acid-α(1→4))-α-L-idopyranoside Uronic Acid;
Ethoxy-2-aminoethoxyl-O-((2,4-O-disulfonato)-α-L-idopyranosyl Uronic Acid-α(1→4)(2-sulfonato))-α-L-idopyroside Uronic Acid;
Ethoxy-2-aminoethoxyl-O-(α-L-idopyranosyl Uronic Acid-α(1→4)-α-L-idopyranosyl Uronic Acid-α(1→4))-α-L-idopyranoside Uronic Acid;
Ethoxy-2-aminoethoxyl-O-((4-O-sulfonato)-α-L-idopyranosyl Uronic Acid-α(1→4)-L-idopyranosyl Uronic Acid-α(1→4))-α-L-idopyranoside Uronic Acid;
Ethoxy-2-aminoethoxyl-O-(2,4-O-disulfonato)-α-L-idopyranosyl uronic acid-α(1→4)(2-O-sulfonato)-α-L-idopyranosyl uronic acid-α(1→4)(2-O-sulfonato)-α-L-idopyranoside uronic acid;
Ethoxy-2-aminoethoxyl-O-(α-L-idopyranosyl uronic acid-α(1→4)-α-L-idopyranosyl uronic acid-α(1→4)-α-L-idopyranosyl uronic acid-α(1→4))-α-L-idopyranoside uronic acid;
Ethoxy-2-aminoethoxyl-O-(4-O-sulfonato)-α-L-idopyranosyl Uronic Acid-α(1→4)-α-L-Idopyranosyl Uronic Acid-α(1→4)-α-L-idopyranosyl Uronic Acid-α(1→4)-α-L-idopyranoside Uronic Acid;
Ethoxy-2-aminoethoxyl-O-((2-4-O-disulfonato)-α-L-idopyranosyl Uronic Acid-α(1→4)(2-O-sulfonato)-α-L-idopyranosyl Uronic Acid-α(1→4)(2-O-sulfonato)-α-L-idopyranosyl Uronic Acid-α(1→4)(2-O-sulfonato))-α-L-idopyranoside Uronic Acid;
3-Aminopropyl-O-[(2-amino-2-deoxy-α-D-glucopyranosyl)-(1→4)-O-(2-O-sulfonato-α-L-idopyranosyluronate)-(1→4)-O-(2-amino-2-deoxy-α-D-glucopyranosyl)]-(1→4)-O-2-O-sulfonato-α-L-idopyranosiduronate;
3-Aminopropyl-O-[(2-acetamido-2-deoxy-α-D-glucopyranosyl)-(1→4)-O-(2-O-sulfonato-α-L-idopyranosyluronate)-(1→4)-O-(2-acetamido-2-deoxy-α-D-glucopyranosyl)]-(1→4)-O-2-O-sulfonato-α-L-idopyranosiduronate;
3-Aminopropyl-O-[(2-amino-2-deoxy-α-D-glucopyranosyl)-(1→4)-O-(α-L-idopyranosyluronate)-(1→4)-O-(2-amino-2-deoxy-α-D-glucopyranosyl)]-(1→4)-O-α-L-idopyranosiduronate;
3-Aminopropyl-O-[(2-acetamido-2-deoxy-α-D-glucopyranosyl)-(1→4)-O-(α-L-idopyranosyluronate)-(1→4)-O-(2-acetamido-2-deoxy-α-D-glucopyranosyl)]-(1→4)-O-α-L-idopyranosiduronate;
3-Aminopropyl-O-[(2-amino-6-O-sulfonato-3-O-sulfonato-2-deoxy-α-D-glucopyranosyl)-(1→4)-O-(α-L-idopyranosyluronate)-(1→4)-O-(2-amino-6-O-sulfonato-3-O-sulfonato-2-deoxy-α-D-glucopyranosyl)]-(1→4)-O-α-L-idopyranosiduronate;
3-Aminopropyl-O-[(2-amino-3-O-sulfonato-2-deoxy-α-D-glucopyranosyl)-(1→4)-O-(2-O-sulfonato-α-L-idopyranosyluronate)-(1→4)-O-(2-amino-3-O-sulfonato-2-deoxy-α-D-glucopyranosyl)]-(1→4)-O-2-O-sulfonato-α-L-idopyranosiduronate;
3-Aminopropyl-O-[(2-amino-6-O-sulfonato-2-deoxy-α-D-glucopyranosyl)-(1→4)-O-(2-O-sulfonato-α-L-idopyranosyluronate)-(1→4)-O-(2-amino-6-O-sulfonato-2-deoxy-α-D-glucopyranosyl)]-(1→4)-O-2-O-sulfonato-α-L-idopyranosiduronate;
3-Aminopropyl-O-[(2-acetamido-6-O-sulfonato-2-deoxy-α-D-glucopyranosyl)-(1→4)-O-(2-O-sulfonato-α-L-idopyranosyluronate)-(1→4)-O-(2-acetamido-6-O-sulfonato-2-deoxy-α-D-glucopyranosyl)]-(1→4)-O-2-O-sulfonato-α-L-idopyranosiduronate;
3-Aminopropyl-O-[(2-acetamido-6-O-phosphonato-2-deoxy-α-D-glucopyranosyl)-(1→4)-O-(2-O-phosphonato-α-L-idopyranosyluronate)-(1→4)-O-(2-acetamido-6-O-phosphonato-2-deoxy-α-D-glucopyranosyl)]-(1→4)-O-2-O-phosphonato-α-L-idopyranosiduronate;
3-Aminopropyl-O-[(2-amino-3-O-sulfonato-2-deoxy-α-D-glucopyranosyl)-(1→4)-O-(α-L-idopyranosyluronate)-(1→4)-O-(2-amino-3-O-sulfonato-2-deoxy-α-D-glucopyranosyl)]-(1→4)-O-α-L-idopyranosiduronate;
3-Aminopropyl-O-[(2-amino-6-O-sulfonato-2-deoxy-α-D-glucopyranosyl)-(1→4)-O-(α-L-idopyranosyluronate)-(1→4)-O-(2-amino-6-O-sulfonato-2-deoxy-α-D-glucopyranosyl)]-(1→4)-O-α-L-idopyranosiduronate;
3-Aminopropyl-O-[(2-acetamido-6-O-sulfonato-2-deoxy-α-D-glucopyranosyl)-(1→4)-O-(α-L-idopyranosyluronate)-(1→4)-O-(2-acetamido-6-O-sulfonato-2-deoxy-α-D-glucopyranosyl)]-(1→4)-O-α-L-idopyranosiduronate;
3-Aminopropyl-O-[(2-acetamido-3-O-sulfonato-2-deoxy-α-D-glucopyranosyl)-(1→4)-O-(α-L-idopyranosyluronate)-(1→4)-O-(2-acetamido-3-O-sulfonato-2-deoxy-α-D-glucopyranosyl)]-(1→4)-O-α-L-idopyranosiduronate;
3-Aminopropyl-O-[(2-acetamido-6-O-phosphonato-2-deoxy-α-D-glucopyranosyl)-(1→4)-O-(α-L-idopyranosyluronate)-(1→4)-O-(2-acetamido-6-O-phosphonato-2-deoxy-α-D-glucopyranosyl)]-(1→4)-O-(α-L-idopyranosyluronate)];
3-Aminopropyl-O-[(2-deoxy-2-acetamido-α-D-glucopyranosyl)-(1→4)-O-(β-D-glucopyranosyluronate)-(1→4)-O-(2-deoxy-2-acetamido-α-D-glucopyranosyl)-(1→4)-O-(β-D-glucopyranosyluronate)];
3-Aminopropyl-O-[(2-deoxy-2-acetamido-6-O-sulfonate-α-D-glucopyranosyl)-(1→4)-O-(β-D-glucopyranosyluronate)-(1→4)-O-(2-deoxy-2-acetamido-6-O-sulfo-α-D-glucopyranosyl)-(1→4)-O-(β-D-glucopyranosyluronate)];
3-Aminopropyl-O-[(2-deoxy-2-acetamido-3-O-sulfonate-α-D-glucopyranosyl)-(1→4)-O-(β-D-glucopyranosyluronate)-(1→4)-O-(2-deoxy-2-acetamido-3-O-sulfo-α-D-glucopyranosyl)-(1→4)-O-(β-D-glucopyranosyluronate)];
Cholesteryl-O-((2,4-O-disulfonato)-α-L-idopyranosyl uronic acide-α(1→4)(2-O-sulfonato))-α-L-idopyranoside uronic acid;
Cholesteryl-O-((2,4-O-disulfonato-3-O-benzyl-)-α-L-idopyranosyl uronate-α(1→4)(2-O-sulfonato-3-O-benzyl)-α-L-idopyranosyl uronate-α(1→4)(2-O-sulfonato-3-O-benzyl))-α-L-idopyranoside urinate;
Cholesteryl-O-((2,4-O-disulfonato)-α-L-idopyranosyl uronate-α(1→4)(2-O-sulfonato)-α-L-idopyranosyl uronate-α(1→4)(2-Osulfonato)-α-L-idopyranosyl uronate-α(1→4)(2-Osulfonato))-α-L-idopyranoside urinate;
Cholestanyl-O-((2,4,6-O-trisulfonato)-α-L-idopyranosyl-α(1→4)(2,6-O-disulfonato))-α-L-idopyranoside;
Cholestanyl-O-((2,4,6-O-trisulfonato)-α-L-idopyranosyl-α(1→4)(2,6-O-disulfonato)-α-L-idopyranosyl-α(1→4)(2,6-O-disulfonato))-α-L-idopyranoside; and
Cholesteryl-O-((2,4,6-O-trisulfonato)-α-L-idopyrnosyl-α(1→4)(2,6-O-disulfonato-3-O-benzyl 3)-α-L-idopyrnosyl-α(1→4)(2,6-O-disulfonato)-α-L-idopyrnosyl-α(1→4)(2,6-O-disulfonato))-α-L-idopyranose.

8. A pharmaceutical composition comprising a compound as claimed in claim 1 and one or more pharmaceutically acceptable excipients.

9. The pharmaceutical composition as claimed in claim 8, wherein the composition is in the form of pills, tablets, coated tablets, capsules, granules or elixirs.

10. A method of treating cancer comprising administering a therapeutically effective amount of a compound or pharmaceutically acceptable salt thereof as claimed in claim 1 to a subject in need thereof.

11. (canceled)
